(19) United States
(12) Patent Application Publication
(10) Pub. No.: US 2023/0253072 A1
(43) Pub. Date: Aug. 10, 2023

(54) COMPUTER IMPLEMENTED METHODS FOR DETECTING ANALYTES IN IMMUNOASSAYS

(71) Applicant: INVITROGEN BIOSERVICES INDIA PRIVATE LIMITED, Bengaluru (IN)

(72) Inventors: Sriram PARAMESWARAN, Bengaluru (IN); Rajeev PANDEY, Bengaluru (IN); Neetu SAIN, Bengaluru (IN)

(21) Appl. No.: 18/148,932

(22) Filed: Dec. 30, 2022

Related U.S. Application Data
(60) Provisional application No. 63/386,814, filed on Dec. 9, 2022.
(60) Provisional application No. 63/295,160, filed on Dec. 30, 2021.

(51) Int. Cl. G16B 40/10; G16B 40/20; G06T 7/00

(52) U.S. Cl. CPC G16B 40/10; G16B 40/20; G06T 7/0012; G06T 2207/20081; G06T 2207/20084

(57) ABSTRACT

Provided are computer implemented systems and methods for detecting one or more analytes in an immunoassay. Embodiment systems and methods of the disclosure can train a machine learning network using immunoassay reference data on an analyte and output a set of target experimental input parameters to detect the analyte. A machine learning network can utilize the received data to generate target parameters, and can develop an immunoassay parameter set to detect and/or quantify an analyte. Computer implemented systems and methods of the disclosure can also generate troubleshooting assistance outputs for immunoassays.

### CROSS-REFERENCE TO RELATED APPLICATIONS

The present application claims the benefit of U.S. Provisional Application No. 63/386,814, filed Dec. 9, 2022, and U.S. Provisional Application No. 63/295,160, filed Dec. 30, 2021, each of which is incorporated herein by reference in its entirety.

### TECHNICAL FIELD

Embodiments of the present disclosure generally relate to computer implemented methods for detecting and/or quantifying analytes, and more particularly, detecting and/or quantifying analytes by immunoassay methods.

### BACKGROUND

Immunoassay experimental design techniques often utilize empirical testing methods to identify optimal parameters and to troubleshoot issues. These empirical methods, such as trial and error testing, can be inefficient with respect to time and resources. For example, some immunoassay methods, such as immunoblotting or Western Blot methods, are not easily scalable due to pre-optimization limitations, and optimization determinations quickly become more complex and time-consuming when multi-variable optimizations are introduced.

A key factor in optimizing immunoassay performance lies in determining an optimal reagent and experimental parameters to ensure a successful detection in the immunoassay. The reagent choice, along with the analyte or protein concentration and antibody dilution, are considerations for ensuring a successful detection in immunoassay experiments. These optimal variable values are often determined experimentally.

Although optimization techniques such as using empirical values based on best guesses and/or testing a series of values for individual experiments are very time and cost intensive, they are especially common with Western Blot experiments, and workflows involving instruments such as imagers and blotters, such as, but not limited to, iBright FL1500™, iBlot™, and chemiluminescent detection technologies.

Other factors, such as optimization of immunoassay parameters based on analyte abundance, antibody binding variation and choice sensitivity detection substrate can cause additional noise in the experimental data, and lead to misleading data, errors, and customer dissatisfaction. Further, there may also be noise when detecting analytes because of changing variables in protein preparation, protein forms, and experimental parameters when setting up and performing an immunoassay.

### SUMMARY

Embodiments of the present disclosure relate to computer implemented systems and methods for detecting one or more analytes in an assay, such as an immunoassay. Embodiment systems and methods of the disclosure can train a machine learning network using immunoassay reference data on an analyte and output a set of target experimental input parameters to detect the analyte. A machine learning network can utilize the received data to generate target parameters, and to develop or generate an immunoassay parameter set to detect and/or quantify an analyte. Computer implemented systems and methods of the disclosure can also generate troubleshooting assistance outputs for immunoassays.

Embodiments relate to computer-implemented systems and methods for generating and outputting target parameters, such as but not limited to target experimental input parameters, for immunoassays, and/or to detect one or more analytes in an immunoassay, and/or to operate one or more immunoassay support systems and apparatuses. In some embodiments, generating and outputting target experimental input parameters are to improve or optimize detection of analyte by an immunoassay. Some embodiments utilize semi-automated approaches to extract data from standard immunoblot data, train machine learning model using validated immunoblot data, and apply reference data sets to generate and/or output and/or predict target experimental parameters for immunoassays. Target parameters can include input variables, such as experimental input variables such as a sample concentration, a protein concentration, a detection reagent, and a target loading concentration. Target loading concentrations can assist in achieving a desired result, and can be based on a type of experiment, generated with respect to a sample (e.g., samples comprising one or more analytes, samples suspected of comprising one or more analytes), a specific analyte, and the like.

In some embodiments, systems and methods for detecting one or more analytes in an assay, such as an immunoassay, comprise: training a machine learning network using immunoassay reference data comprising sets of reference input parameters and corresponding reference analyte detection data; receiving a set of experimental input parameters, wherein the experimental input parameters comprise an identifier of an analyte; applying the machine learning network to the immunoassay reference data to identify target parameters based on the analyte; determining an immunoassay parameter set based on the target parameters, wherein the immunoassay parameter set comprises a loading concentration for at least one of the analyte and a detection reagent.

In various embodiments, a reference data set comprises experimental data from large-scale antibody validation datasets. In some examples, large-scale datasets can span over 25000 data points on a wide array of cell and tissue sets. Such data can be manually curated and include classified data demonstrating detection of proteins from cell or tissue lysates loaded across a variety of loading concentrations, detection reagents and antibody affinities.

Embodiments can further utilize machine learning and statistical learning models to generate and output target parameters and/or to guide users on target loading and reagent selections. Results can therefore provide insight on cause-effect variables to optimize immunoassay performance, and generate recommendations for reagent selection, blocking agents, dilution ranges, which can include sample dilution ranges, antibody dilution ranges, and reagent dilution ranges, for example, protein load, and detection reagents, among others.

Embodiments of the machine learning model can uniquely integrate key reference data on cell and tissue protein abundances, such as abundances for a sample type, with training data sets on experimental performance data across a wide set of immunoassays, and further utilize experimental data with antibodies from multiple clonalities and species-specific backbones. The techniques can also utilize datasets with target binding affinities, loading values, multiple detection sensitivities, and cell lines to derive, for example, output values such as binding variations at each protein abundance value.

Such techniques can be applied in a clinical, research, academic, or diagnostic setting, among others. In various embodiments, systems and methods can be executed on a computing device with a graphical user interface, such as a web tool, application, or other display. Such implementations can execute one or more of: an experiment design tool and recommender for single plex assays, a troubleshooting feature, a feature extension on experimental tools, a user cloud account, a trouble-shooting module, an optimization module, a licensed executable for multiplex design, a feature functionality with instrument control, and an analysis software for multiplex assays. Implementations can further include an automated interface, which can simulate detection of immunoassay experiments, and analyze theoretical experimental outputs.

Accordingly, systems and methods of the disclosure can provide information to end-users on target experimental parameters for target analyte detection and quantification. Exemplary analytes include but are not limited to haptens, hormones, nucleic acids, peptides, modified peptides, proteins, or modified form of any of the foregoing analytes. Tools for design of quantitative protein detection can be automated and, in accordance with other embodiments, determine the target loading values for a protein lysate for non-saturating signal. Various computational selection scheme for target experimental designs, can include an experimental design selector, and codes for cell line selection.

Additional advantages and aspects of the disclosed technology include informing the end-user through a software interface of a target protein load for detection and quantification of protein in an immunoblot assay, such as a Western Blot assay. Outputs can inform the range of detection to provide a recommendation on antibody concentration and detection reagent selection for an optimal detection of a protein, such as detection within a target range. In embodiments, the target range can be defined based on one or more factors, including but not limited to user selection, a range to achieve a certain result, such as visibility, identification or the analyte, or other defined criteria. As such, these tools can inform users of optimal variables for the selection and design of multiplex protein detection on immunoassay, and assist experiment design, optimization, and troubleshooting.

Embodiments of the disclosed technology also include manual and automated image analysis tools to extract protein detection values from immunoassay and immunoblot data. Such embodiments can match protein detection to reference values of protein abundances or transcript abundance. Systems can apply methods to cleanse and fit data to exclude outlier data on protein lability, and classify data on variables such as antibody clonality, detection sensitivity, loading concentrations, and protein abundance across a range of cell lines. Data sets can be modeled, trained, and tested using one or more statistical models, machine learning models, or a combination thereof.

In a specific example, embodiments of the disclosed technology can extract protein detection information from a standard immunoblot experiment, such as a Western Blot. Embodiments can model protein abundances, clonality, detection, and select multiplex protein detection and target load-detection values.

A computer-implemented method for analyte detection in immunoassays, can comprise: receiving immunoassay reference data comprising sets of reference input parameters and corresponding reference analyte detection data, receiving a set of experimental input parameters, wherein the experimental input parameters comprise an analyte, applying a machine learning network to the immunoassay reference data to identify target parameters based on the analyte, and determining an immunoassay parameter set based on the target parameters, wherein the output immunoassay parameter set comprises a loading concentration for at least one of the analyte and a detection reagent. In some embodiments, a loading concentration for at least one of the analytes comprises a loading concentration of a sample comprising the analyte. In some embodiments, a loading concentration for at least one of the analytes comprises a loading concentration of a sample comprising the analyte where the sample can be in a sample buffer. In some embodiments, a loading concentration for at least one of the analytes comprises a loading concentration of the analyte comprised in a buffer or solution or a mixture.

In various embodiments, the immunoassay reference data comprises at least one of Western blot data, a multiplex western blot capture, quantitative data, the quantitative data optionally representative of a relative abundance of a protein, categorical data, protein detection data, and immunocytometry data.

Training input parameters can comprise one or more of: user input, a type of analyte, a type of protein, a clonality, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters (e.g., gel composition gel porosity, and the like), analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody concentration/dilution, a binding affinity, an antibody isoform specificity, a backbone type, a protein stability, a protein lability, a detection label, an enzyme, a detection multiplicity or a detection sensitivity. In embodiments, training input parameters can include user input. In singleplex experiments, for example, training input can include a type of protein, a cell line, a lysate concentration, and an antibody dilution. In multiplex experiments and troubleshooting experiments, training input can include user input indicative of a variable such as a set of proteins. In modeling operations, training input can include a set of ranges for variables. The variables can relate to one or more of parameters related to the experiment, a desired input or output, and other information relating to the immunoassay experiment or apparatus.

An immunoassay parameter set can be indicative of an output or prediction or generation of one or more variables, settings, and experimental inputs for executing an immunoassay experiment. An immunoassay parameter set can comprise, for example, one or more of a cell line, an analyte loading range, a target protein, detection data, a lysate type, a lysate loading concentration, an antibody clonality, an antibody type, an antibody binding site, an antibody dilution range, a sample dilution range, a reagent dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte wherein in some embodiments the analyte is comprised in a sample, and/or is comprised in a buffer, a sample buffer, a solution, and/or in a mixture, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type, a generated or outputted or recommended detection reagent, an antibody type, an antibody loading concentration/dilution, a protein lysate concentration, a target cell line, a target protein source, an analyte mass, a transfer condition, a validation flag, and a predicted analyte localization. In embodiments, analytes can be located in several places, for example, an analyte can be secreted from a cell; present in the extracellular fluid; expressed recombinantly and targeted to non-native location; present intracellularly in one or more than one cellular compartment; fractioned biochemically, and/or resolved on biophysical/biochemical properties (such as in western blot or iso-electric focusing)

A system for detecting one or more analytes in an immunoassay, comprising: at least one computing device comprising a processor and at least one memory storing instructions that when executed by the processor, causes the computing device to: receive immunoassay reference data comprising sets of input parameters and corresponding analyte detection data; receive a set of experimental input parameters, wherein the experimental input parameters comprise an identifier of an analyte and a clonality; apply a machine learning network to the immunoassay reference data to identify target parameters based on the analyte; determine an immunoassay parameter set based on the target parameters; and provide the immunoassay parameter set, wherein the immunoassay parameter set comprises a loading concentration for at least one of the analyte comprised in a sample, a buffer, a solution, and/or a mixture and a detection reagent.

In some embodiments, in a system of the disclosure, the at least one memory stores instructions that when executed by the processor, further causes the computing device to: extract relevant immunoassay reference data based on the experimental input parameters; determine a relationship between the experimental input parameters and corresponding reference analyte detection data; and determine or generate target parameters for detecting the analyte.

In some embodiments, in a system of the disclosure, the at least one memory stores instructions that when executed by the processor, further causes the computing device to: classify the relevant immunoassay reference data into variables; and apply a statistical model to determine relationships between two or more variables; and train the machine learning network using the relationships between the two or more variables.

In some embodiments, in a system of the disclosure, the instructions further cause the computing device to extract immunoassay reference data from images representative of experimental immunoassay data.

A system of the disclosure, in some embodiments, further comprises a user interface, and the user interface comprises at least one of: an instrument console, a web tool, a graphical user interface, and a display on a computing device.

In some embodiments, the disclosure comprises a non-transitory computer-readable medium storing instructions that, when executed by one or more processors, cause a device to perform one of the methods described herein.

In some embodiments, computer-implemented methods of the disclosure are for operating an immunoassay instrument support apparatus, and comprise: receiving immunoassay reference data comprising sets of reference input parameters and corresponding reference analyte detection data; receiving a set of experimental input parameters, the experimental input parameters comprising at least one variable related the immunoassay experiment; receiving information indicative of an issue with at least one of: the immunoassay reference data, an experimental input parameter, and a result of immunoassay experiment; applying a machine learning network to the immunoassay reference data to identify target parameters based on the issue; and determining an immunoassay parameter set to resolve based on the target parameters.

Exemplary issues include but are not limited to at least one of an analyte detection issue or an immunoassay reference data extraction issue. In some embodiments, an issue relates to extraction of immunoassay reference data from images indicative of experimental immunoassay data.

In some embodiments, a computer-implemented method of the disclosure, is for operating an immunoassay instrument support apparatus, and comprises: receiving immunoassay reference data comprising sets of reference input parameters and corresponding reference analyte detection data; receiving a set of experimental input parameters; receiving at least one target variable for to the immunoassay experiment; applying a machine learning network to the immunoassay reference data to identify target parameters based on the target variable; and determining an immunoassay parameter set for obtaining the target variable based on the target parameters, wherein the target variable is an analyte. In embodiments, set of experimental input parameters represent a method of experimentation. Non-limiting methods of experimentation include a Western Blot method, an immunoblotting method, a transfer method, and method utilizing protein gel.

In some embodiments, a computer-implemented method of the disclosure, for operating an immunoassay instrument support apparatus for immunoblotting, comprises: receiving immunoblot reference data comprising sets of reference input parameters and corresponding reference analyte detection data; receiving a set of experimental input parameters, wherein the experimental input parameters comprise an identifier of an analyte; applying a machine learning network to the immunoblot reference data to identify target parameters based on the analyte; and determining an immunoassay parameter set based on the target parameters, wherein the immunoassay parameter set comprises a loading concentration for at least one of the analyte comprised in a sample, a buffer, a mixture and/or a solution and a detection reagent.

In some embodiments, a computer-implemented method of the disclosure, for operating an immunoassay instrument support apparatus for immunoblotting, comprises: receiving immunoblot reference data comprising sets of reference input parameters and corresponding reference analyte detection data; receiving a set of experimental input parameters, wherein the experimental input parameters comprise an identifier of an analyte; applying a machine learning network to the immunoblot reference data to identify target parameters based on the analyte; and determining an immunoassay parameter set based on the target parameters, wherein the immunoassay parameter set comprises a loading concentration for at least one of the analyte comprised in a sample, a buffer, a mixture and/or a solution and a detection reagent.

In some embodiments of the disclosure, a method for operating an immunoassay instrument support apparatus comprises: receiving immunoassay reference data comprising sets of reference input parameters and corresponding reference analyte detection data; receiving a set of experimental input parameters, wherein the experimental input parameters comprise an identifier of an analyte, and single cell information; applying a machine learning network to the immunoassay reference data to identify target parameters based on the identifier of an analyte; and determining an immunoassay parameter set for detecting multiple proteins in the single cell based on the target parameters, wherein the immunoassay parameter set comprises a loading concentration for at least one of the analyte comprised in a sample and a detection reagent. Non-limiting examples of a single cell include one or more of: a cell line or a cell lineage identified by microscopic morphology, sorted/tracked using one or more than one biomarker tags, a cell expressing a fluorescent reporter or a cell that is cultured as isolated primary or maintained cell-lines. A non-limiting example of single cell information can include one or more of: a transcript abundance, an analyte (such as a protein, nucleic acid, etc.) localization information, or proteome data on analyte/protein abundances, such as mass-spectroscopy investigation on analyte/protein abundance, protein post-translation modification such as glycan modification, phosphorylation, ubiquitination, SUMOlyation, lipid anchor etc.

Some embodiments describe computer-implemented methods for operating an immunoassay instrument support apparatus for flow-based immunoassays, comprising: receiving immunoassay reference data comprising sets of reference input parameters and corresponding reference analyte detection data, wherein the immunoassay reference data comprises at least one set of flow-based immunoassay data; receiving a set of experimental input parameters, wherein the experimental input parameters comprise an identifier of an analyte, and single cell information; applying a machine learning network to the immunoassay reference data, including the flow-based immunoassay data to identify target parameters based on the analyte; and determining an immunoassay parameter set for detecting multiple proteins in the single cell based on the target parameters, wherein the immunoassay parameter set comprises a loading concentration for at least one of the analyte comprised in a sample and a detection reagent.

In some embodiments, single cell information could be data for surface markers, secreted analytes or intracellular markers. Such single cell information can be derived from flow-cytometry, image analysis or protein localization studies.

Exemplary non-limiting flow assays include assays where a set of analytes/proteins with varying abundances is to be profiled, and dye intensity to be matched is inversely proportional to analyte/protein abundance values. In such examples, one embodiment is where a cell-type expresses a set of analytes (proteins or protein-modification targeting antibodies) and comprise identifying a cell that is suitable for concurrent investigation of the set of user selected analytes in an experiment. Another embodiment is to select of a set of antibodies and dyes that lead to optimal spectral compensation based on dye intensity and expected analyte abundances.

Accordingly, embodiments of the disclosed technology include systems and methods for one or more of: applying machine learning to improve detection in immunoassays; and/or entering input parameters and receiving an output of target immunoassay parameter sets via, e.g., a user interface, applying various techniques to a computer, web tool, or other hardware device; and/or troubleshooting issues. Such techniques can be applied to Western Blot methods, bead-based assays, profiling of multiple analytes, detection of multiple analytes, profiling of multiple proteins, detection of multiple proteins, analyte imaging, protein imaging, flow cytometry-based detections, and fluorescent and dye detection.

### BRIEF DESCRIPTION OF THE DRAWINGS

The summary, as well as the following detailed description, is further understood when read in conjunction with the appended drawings. For the purpose of illustrating the disclosed subject matter, there are shown in the drawings exemplary embodiments of the disclosed subject matter, however, the disclosed subject matter is not limited to the specific methods, compositions, and devices disclosed. In addition, the drawings are not necessarily drawn to scale. In the drawings:

FIG. 1 illustrates an example flowchart for providing a recommendation in accordance with embodiments of the disclosed technology.

FIG. 2 illustrates an example flowchart for predicting target parameters for analyte detection in accordance with embodiments of the disclosed technology.

FIG. 3 illustrates a model relationship between protein abundances, clonality, and detection in accordance with embodiments of the disclosed technology.

FIG. 4 illustrates an immunoassay dose response curve in accordance with embodiments of the disclosed technology.

FIG. 5 illustrates a linear and logistic dose response model in accordance with embodiments of the disclosed technology.

FIG. 6 illustrates an implementation of a machine learning model in accordance with embodiments of the disclosed technology.

FIG. 7 illustrates a multiplex protein detection flowchart in accordance with embodiments of the disclosed technology.

FIG. 8 illustrates an example user interface output in accordance with embodiments of the disclosed technology.

FIG. 9 illustrates an example user interface output in accordance with embodiments of the disclosed technology.

FIG. 10 illustrates an immunoassay experiment in accordance with embodiments of the disclosed technology.

FIG. 11 illustrates an immunoassay experiment in accordance with embodiments of the disclosed technology.

FIG. 12 illustrates an immunoassay experiment in accordance with embodiments of the disclosed technology.

FIG. 13 illustrates an immunoassay experiment in accordance with embodiments of the disclosed technology.

FIG. 14 illustrates an immunoassay experiment in accordance with embodiments of the disclosed technology.

FIG. 15 illustrates a prediction based on target abundance in accordance with embodiments of the disclosed technology.

FIG. 16 illustrates a computing system in accordance with embodiments of the disclosed technology.

FIG. 17 illustrates a method for identifying target parameters of an experiment based on an analyte with embodiments of the disclosed technology.

FIG. 18 illustrates a method for determining cell line recommendations for use in an immunoassay experiment with embodiments of the disclosed technology.

FIG. 19 illustrates a method for ranking cell line recommendations for use in an immunoassay experiment with embodiments of the disclosed technology.

FIG. 20 illustrates a method for determining cell line recommendations for use in an immunoassay experiment with embodiments of the disclosed technology.

FIG. 21 illustrates an example of a user interface that receives an analyte of interest and displays cell line recommendations for an immunoassay experiment with embodiments of the disclosed technology.

FIG. 22 illustrates an example of a user interface that displays the ranking order of cell line recommendations for an immunoassay experiment with embodiments of the disclosed technology.

FIG. 23 illustrates an example of a user interface that recommends experiment recommendations for specific features of an immunoassay experiment with embodiments of the disclosed technology.

FIG. 24 illustrates an example of a user interface that displays recommendations for specific features of an immunoassay experiment with embodiments of the disclosed technology.

FIG. 25 is a method for determining the migration of an analyte in an immunoassay with embodiments of the disclosed technology.

FIG. 26 illustrates an example diagram of an immunoassay experiment with embodiments of the disclosed technology.

FIG. 27 illustrates a method for a system that may be used to determine the migration of a band in an immunoassay experiment with embodiments of the disclosed technology.

FIG. 28 illustrates an example of a user interface for a user to load immunoblot data for analysis of an immunoblot and analyte migration with embodiments of the disclosed technology.

FIG. 29A-B illustrates an example of a user interface that receives experimental data for an immunoassay experiment with embodiments of the disclosed technology.

FIG. 30A-B illustrates an example of a user interface that displays the results of analysis on an immunoassay experiment with embodiments of the disclosed technology.

FIG. 31 illustrates an example of an immunoblot with multiple bands of interest being reported with embodiments of the disclosed technology.

Aspects of the present invention will be described with reference to the accompanying drawings. The drawing in which an element first appears is typically indicated by the leftmost digit(s) in the corresponding reference number.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the claimed subject matter.

In the detailed description that follows, references to “one aspect”, “an aspect”, “an example aspect”, etc., indicate that the aspect described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is submitted that it is within the knowledge of one skilled in the art to effect such feature, structure, or characteristic in connection with other aspects whether or not explicitly described. The words “aspect” and “embodiment” may be used interchangeably.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. It is to be understood that the terminology used herein is for the purpose of describing particular aspects only and is not intended to be limiting.

It is to be appreciated that certain features of the disclosed subject matter which are, for clarity, described herein in the context of separate embodiments, can also be provided in combination in a single embodiment. Conversely, various features of the disclosed subject matter that are, for brevity, described in the context of a single embodiment, can also be provided separately or in any sub combination. Further, any reference to values stated in ranges includes each and every value within that range. Any documents cited herein are incorporated herein by reference in their entireties for any and all purposes.

An immunoassay may be used to detect the presence and/or expression level of a particular analyte (e.g., peptide, polymer, protein, etc.) in a sample. Immunoassays are test experiments that use a tagging molecule to detect and quantitate a substance, specifically an analyte, in a test sample. In some aspects, the tagging molecule may be an antibody, oligonucleotide, or an analyte stain (e.g. Coomassie, silver stain, or the like). Often, the immunoassay uses electrophoresis, such as gel electrophoresis, to detect and quantitate a specific analyte. The output of an immunoassay may be an image, referred to as an immunoblot. It is expected that the analyte will migrate through the gel (or other membrane depending on the type of electrophoresis used) based on a function of mass (molecular weight), so that analytes of different mass can be identified as separate bands within an immunoblot. Such techniques may be used, for example, to identify and quantitate proteins during protein preparation.

Embodiments of the disclosure describe systems and methods for detecting one or more analytes in an assay, such as an immunoassay, comprising: training a machine learning network using immunoassay reference data comprising sets of reference input parameters and corresponding reference analyte detection data; receiving a set of experimental input parameters, wherein the experimental input parameters comprise an identifier of an analyte; applying the machine learning network to the immunoassay reference data to identify target parameters based on the analyte; determining an immunoassay parameter set based on the target parameters, wherein the immunoassay parameter set comprises a loading concentration for at least one of the analyte and a detection reagent. Embodiment methods of the disclosure comprise computer-implemented methods.

Embodiments relate to systems and methods for generating and predicting target parameters, such as but not limited to target experimental input parameters, for immunoassays, and/or to detect one or more analytes in an immunoassay, and/or to operate one or more immunoassay support systems and apparatuses. In some embodiments, generating and predicting target experimental input parameters are to optimize assay conditions for an immunoassay. Some embodiments utilize semi-automated approaches to extract data from standard immunoblot data, train a machine learning model using validated immunoblot data, and apply reference data sets to predict target experimental parameters for immunoassays. Target parameters can include input variables, such as experimental input variables such as a sample concentration, a protein concentration, a detection reagent, and a target loading concentration. Target loading concentrations can assist in achieving a desired result, and can be based on a type of experiment, generated with respect to a sample (e.g., samples comprising one or more analytes, samples suspected of comprising one or more analytes), a specific analyte, and the like.

Non-limiting examples of samples having analytes to be detected or quantified by the present methods and systems include: a biological sample, a water sample, an environmental sample, an air sample, a forensic sample, an agricultural sample, a pharmaceutical sample, a food sample. The analyte may be natural or synthetic. A biological sample can be a sample obtained from eukaryotic or prokaryotic sources. Non-limiting examples of eukaryotic sources include mammals, such as a human, a cow, a pig, a chicken, a turkey, a livestock animal, a fish, a crab, a crustacean, a rabbit, a game animal, and/or a murine animal such as rat or mouse. A biological sample may include non-limiting examples such as biological fluids, cells and/or tissues including blood, plasma, cerebrospinal fluids, lymph, bone marrow, nasal fluids/swabs, pharyngeal swab or samples, saliva, urine, feces, a cellular sample (including cells, a single cell, cell lysate, cell components, and/or material derived from a cell), or a tissue.

Embodiments of the disclosed technology can provide an outputting of target parameters (such as target experimental input parameters), to improve analyte detection in assays such as immunoassays. In some embodiments, outputting parameters techniques can utilize semi-automated approaches. Embodiments further comprise methods to extract data from standard immunoblot data, train a machine learning network or model using validated immunoblot data and apply reference data sets to output experimental parameters for target or improved immunoblot assays.

Embodiments in accordance with the disclosed technology include systems and methods for applying machine learning to improve detection in immunoassays; providing or outputting target parameters and based on desired input parameters; providing experimental design parameter output or recommendations; methods for profiling multiple analytes such as but not limited to proteins; methods for detecting multiple analytes, such as but not limited to proteins in single cell experiments; and various applications to Western Blot experiments, flow-based detection, fluorescent dye detection, and tag-count based detection methods.

Accordingly embodiments can uniquely integrate key reference data on cell and tissue wide protein abundances with training data sets on experimental performance data across a wide set of immunoblotting assays; use experimental data with antibodies from multiple clonalities/species specific backbones with target binding affinities to train the model and derive the binding variations at each protein abundance values; and use data from multiple detection sensitivities, loading values and cell lines to model and predict output values. In examples, such key reference data with respect to cell and tissue-wise protein abundances can refer to amounts of cells and proteins used in past experiments, a type of cell or tissue, as discussed herein.

Embodiments can include a validation selector, and codes for cell line selection and automation. Embodiments additionally have the capability to apply a vast training set, with advanced machine learning and AI-based decision models in an easy-to-use interface design and analyze multiplex protein detection.

Multiplex assays, as discussed herein, refer to immunoassays that can measure multiple analytes. Multiplex protein detection therefore refers to detection of multiple proteins. In some aspects, multiplex assays can utilize pull-down techniques, magnetic beads, e.g., for binding antibodies, cells, or lysate preparation. In some aspects, multiplex protein detection can utilize antibodies that are analyzed using IP-MS, secondary antibody enzymes, dye conjugate, primary conjugate dye, or polymer tags. It will be understood that a variety of multiplex assays, proteins, sources, analytes, and samples known in the art, can be used in accordance with embodiments discussed herein.

Accordingly, the disclosed technology is uniquely able to at least predict immunoassay performance and design immunoassays which is helpful in using these technologies in clinical and diagnostic setting, optimize protein quantification and detection, and optimize loading and detection sensitivity for protein quantification.

The disclosed technology also supports experimental planning and design techniques for multiplex protein detection and driving growth in precision cell analysis through, for example, abilities to optimize and troubleshoot an immunoblot assay, determine the target loading of protein lysate for detection in an immunoblot experiment, and plan multiplex western detection in a cell or tissue lysate.

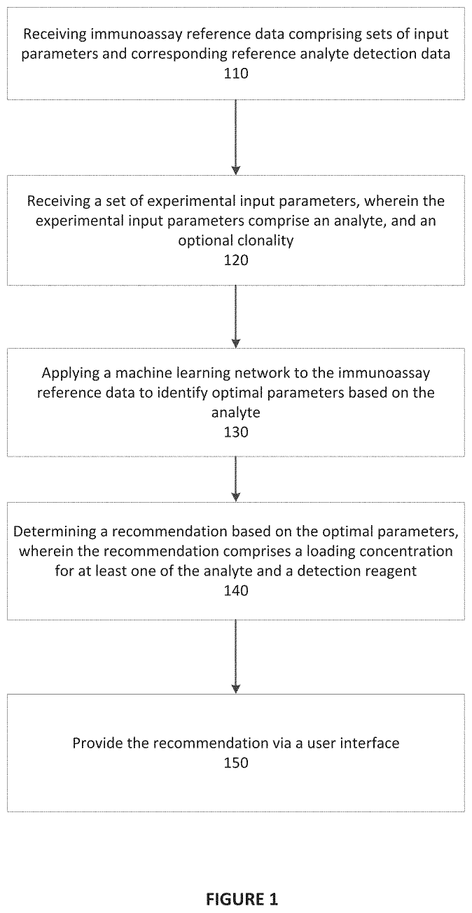

FIG. 1 illustrates an example flowchart for embodiments and models for providing immunoassay design recommendations as discussed herein. In 110, embodiments can receive immunoassay reference data comprising sets of input parameters, which can correspond to reference analyte detection data. Immunoassay reference data can originate from previous experiments, and can comprise results from a set of input parameters. In 120, embodiments can receive a set of experimental input parameters, comprising an identifier of an analyte and an optional clonality. In some aspects, the immunoassay reference data set can include one or more of cell and tissue protein abundance, intensity of a band detected for an antibody that is determined experimentally, protein turn-over and half-life data, protein isoform data, protein compartment localization, post translational modifications, sequence data, and membrane topology data. In some aspects, the immunoassay reference data set can include each of these data types. In some aspects, the immunoassay reference data set can include at least these data types.

In embodiments, an identifier of an analyte can refer to a name of the analyte. The name can be a common name, a scientific or molecular name, a user-defined name, a symbol, a code, or other method of referring to and identifying a type of analyte. In embodiments, a clonality can refer to at least one of a monoclonal analyte recommendation or a polyclonal analyte.

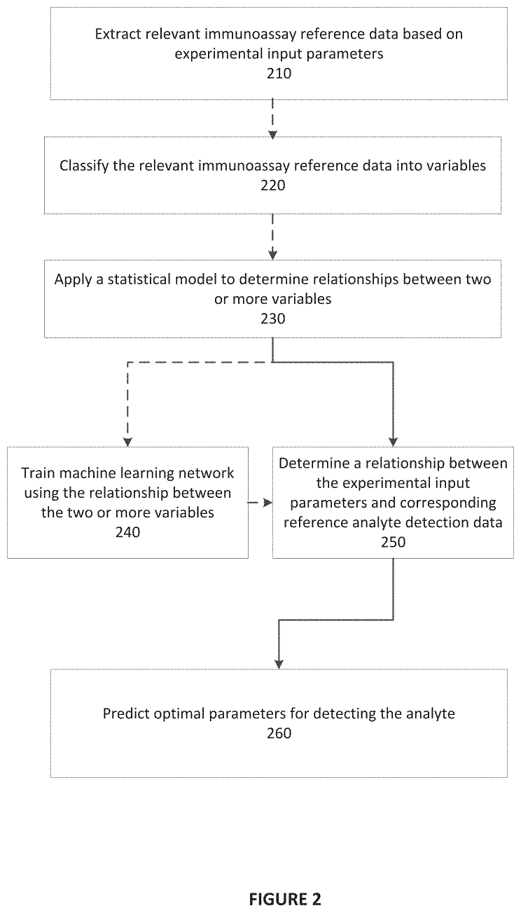

In 130, embodiments can further apply one or more machine learning networks to immunoassay reference data to identify target parameters based on the analyte. An example machine learning methodology that may be used in step 130, according to an embodiment, is further discussed with respect to FIG. 2. Once optimal parameters have been identified, in 140 embodiments can then determine a recommendation based on the target parameters, wherein the recommendation comprises a loading concentration for at least one of the analyte and a detection reagent. In 150, the recommendation can be output on a user interface.

In embodiments, loading concentration for at least one of the analyte can include an analyte comprised in a sample and/or further comprised in a sample buffer, or comprised in a solution, a buffer, or in a mixture, such as a mixture with other sample components including other cellular components, other tissue components or a mixture comprising one or more salts, buffer related components, stabilizing agents, chemicals, preservatives and the like. In embodiments, the loading concentration can relate to an amount of analyte, or other input, for an immunoassay experiment. The loading concentration can vary, based on a type of immunoassay (e.g., immunoblot, flow-based, fluorescent, gel-based, etc.), a desired result, or other experimental factor, as discussed herein.

In embodiments, analytes can comprise at least one of a protein, a hapten, a hormone, a nucleic acid, a peptide, a modified peptide, or modified form of any of the foregoing analytes. In embodiments, the modified peptide can be formed from at least one of a methylation and acetylation.

Embodiments of machine learning models discussed herein can provide multiple utilities, including but not limited to: informing the end-user through a software interface for target protein load for detection and quantification of protein in a western blot assay, informing the range of detection to provide recommendation on antibody concentration, lysate preparation, gel type, detection reagent selection for target detection of protein, and providing tools for selection and design of multiplex protein detection on immunoassay to guide experiment design.

Embodiments can further extract immunoassay training and reference data from images indicative of experimental immunoassay data, receive information indicative of a recommendation type, the recommendation type being at least one of a troubleshooting solution or an experimental design, and update the target parameters based on the immunoassay parameter set, or other recommendation type.

In embodiments, optimal, target parameters can relate to at least one of: an immunoblotting method, such as a Western Blot application, a transfer method (i.e., relating to the immunoassay technique, for example, wet transfer/electroblotting, semi-dry transfer/electroblotting, and dry transfer/electroblotting), and a type of protein gel. Troubleshooting solutions, as discussed herein, can identify one or more of an analyte source, an antibody, and dilution detection information. Dilution detection information can include, but are not limited to, antibody dilution and reagent dilution.

FIG. 2 illustrates an example flowchart for embodiments and models for providing a recommendation and/or prediction for optimal analyte detection parameters. In various embodiments, in 210 systems and methods can extract relevant immunoassay reference data based on experiments input parameters, as discussed herein. In 220, embodiments classify the relevant immunoassay reference data into variables, and in 230 can apply a statistical model to determine relationships between two or more variables.

Some embodiments can optionally, in 240, train machine learning networks and programs using the determined relationships between the two or more variables. Whether or not the training step is implemented, in 250 systems and methods can determine a relationship between the experimental input parameters and corresponding reference analyte detection data, as discussed herein. Accordingly, in 260 embodiments can predict target parameters for detecting the analyte.

Additional embodiments can identify immunoassay reference data related to a sub-class of the analyte, determine one or more sub-class of analyte(s) of interest, and update the target parameters based on the detected sub-class of analytes, wherein the sub-class of analytes is a transmembrane protein, a labile protein, a phosphorylation modification, a glycosylation modification, a post-translational modification, a protein form, a protein isoform, a cleaved variant of protein, or a mutant variant of protein.

It will be appreciated that the type of immunoassay, statistical models, experimental input parameters, variables, target parameters, analytes, and recommendations can be customized to cover a variety of inputs, desired outputs, experiment types, and the like, including but not limited to the examples provided below.

In various embodiments, experimental input parameters can comprise data from a plurality of assays. In embodiments, experimental input parameters can comprise one or more of: a type of analyte (e.g., a hapten, a hormone, a nucleic acid, a peptide, a modified peptide, or modified form of any of the foregoing analytes), a type of protein, a clonality, a cell line, a detection reagent, an antibody concentration, a substrate, a substrate sensitivity, a detection sensitivity, a lysate concentration (such as a lysate of a cell sample or a tissue sample), a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Experimental input parameters related to detection data can further comprise chemical detection data (e.g., chemical substrates), a biochemical detection data (e.g., enzymes), a sequence-based detection, an amplification based detection, and/or fluorescence detection data. Experimental input parameters can further comprise an analyte source, a set of proteins and a cell line, a set of constraints received via a user interface, and defining desired experimental parameters. The generated immunoassay parameter set can comprise determined and/or recommended values for the set of variables, wherein the recommended values are determined to assist in achieving the desired outcome of the immunoassay experiment. In examples, an analyte source can be a protein source, or other biochemical or molecular source for the analyte.

In other embodiments, experimental input parameters can comprise at least one detection protein or target analyte, a set of proteins and a set of constraints received via a user interface, and the target parameters identify recommended values for at least one constraint in the set of constraints. The set of constraints can further comprise at least one of: an available lysate, a cell line, a tissue type, a detection technology, an antibody clonality, a hapten, a hormone, a modified nucleic acid, a peptide, an antibody clonality, a protein, an antigen, analyte size, a protein source, a cell line, a tissue type, a detection sensitivity, loading concentration, a protein abundance, an antibody effect (e.g., visualization, immobilization on a surface or medium, binding effects, etc.), a membrane effect, a blocking effect, an extraction effect, a protein lability, a membrane type, an analyte abundance, an antibody binding variation, an antibody clonality, a binding affinity, an antibody isoform specificity, a backbone type, a detection label, an enzyme, a detection multiplicity or a detection sensitivity.

In embodiments, experimental input parameters can further comprise a set of variables received via a user interface, the issue relates to detection, and the recommendation comprises recommended values for the set of variables. The set of experimental input parameters can represent a method of experimentation, wherein the method of experimentation is a Western Blot method, an immunoblotting method, a transfer method, and method utilizing protein gel.

In various embodiments, immunoassays can include one of many types and designs of immunoassay experiments. Embodiments can utilize, for example, bead-based immunoassay, for example, a multiplex assay, a bead-based immunoassay utilizing panels, or a bead-based immunoassay utilizing activated surfaces panel builder. In other embodiments, a flow-based immunoassay can be a lateral flow immunoassay, a flow assay that uses colored particles, a competitive assay, and the like.

Statistical models discussed herein can comprise at least one of: a cost function, a logistic regression model, a multivariate regression model, a random forest model, a neural network model, and a stochastic gradient model. In embodiments, the machine learning network can apply at least one of a regression model, a decision tree-based training model, and a stochastic gradient descent model.

Target parameters discussed herein can comprise at least one of: a protein, an antigen, analyte size, a protein source, a cell line, a tissue type, a detection sensitivity, loading concentration, a protein abundance, such as a protein abundance in a sample, a protein abundance in a cell, etc., an antibody effect (e.g., visualization, immobilization on a surface or medium, binding effects, etc.), a membrane effect (e.g., a measurable effect on or related to an immunoassay membrane), a blocking effect (e.g., a measurable effect due to a blocking agent, such as an active blocker, passive blocker, special blocking agent, crosslinking blocking agent, etc.), an extraction effect (e.g., resulting from removal of interfering proteins in a sample), a protein lability, a membrane type, an analyte abundance, a lysate, an antibody binding variation, an antibody clonality, a binding affinity, an antibody isoform specificity, a backbone type (e.g., a protein backbone, related to one or more of the analyte or antibody), a detection label, an enzyme, a detection multiplicity or a detection sensitivity, type of gel, type of membrane transferred onto, transfer method, transfer buffer, wash protocols or wash buffer. Immunoassay reference data, as discussed herein, can comprise at least one of Western blot data, a multiplex western blot capture, quantitative data, the quantitative data optionally representative of a relative abundance of a protein, categorical data (e.g., data that can be divided into groups), protein detection data, and immunocytometry data. Immunocytometry data can further comprise at least one of analyte localization data or analyte intensity data. Such data can refer to analyte localization data within at least one of a cell, antibody or sample, and analyte intensity data with respect to at least one of the cell, antibody, or sample. In exemplary scenarios, an analyte can be secreted from a cell; present in the extracellular fluid; expressed recombinantly and targeted to non-native location; present intracellularly in one or more than one cellular compartment; fractioned biochemically—resolved on biophysical/biochemical properties (such as in western blot or iso-electric focusing)

Quantitative data can comprise at least one of an analyte abundance estimate, and analyte detection data from a plurality of experiments utilizing one or more of various loading concentrations, various detection reagents, and various antibody affinities.

In embodiments, immunoassay reference data can be extracted from images representative of experimental immunoassay data.

Recommendations, as discussed herein, can comprise one or more of a cell line, a tissue, an analyte loading range, a target protein, detection data, a lysate type, a lysate loading concentration, a protein clonality, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an optimal range for the loading concentration for at least one of the analyte, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type (e.g., a particle gel, agarose gel, or other gel usable in immunoassay experiments) a recommended detection reagent, an antibody type, an antibody loading concentration, a protein lysate concentration, an optimal cell line, an optimal protein source, an analyte mass, a transfer condition, a validation flag, and a predicted analyte location.

Detection techniques discussed herein can apply to at least one of: chemiluminescence, fluorescence, enzymes, and colorimetric analysis. Other embodiments can include a target range for the loading concentration for at least one of the analyte and the detection reagent. In embodiments, the analyte can be in a sample or a purified form.

An immunoassay performance prediction can include but is not limited to providing at least one of: a recommended analyte, a recommended detection reagent, an antibody concentration, a protein lysate concentration, an antibody type, an optimal cell line, an optimal protein source, an analyte mass, a transfer condition, a validation flag, a predicted analyte location (e.g., an analyte position with respect to a gel or medium on which the immunoassay experiment can be executed, a location relative to one or more samples, etc.), a protein lysate from a cell line source, a type of lysate, an antibody dilution range for optimal detection, an antibody dilution based on a clonality and host backbone type, a type of detection reagent, a type of detection technology, and an optimal detection technology to ensure linearity.

In various embodiments, determining a recommendation for detecting multiple proteins in the single cell can be based on the optimal parameters, wherein the recommendation comprises a loading concentration for at least one of the analyte and a detection reagent. The recommendation can further comprise a type of dye for detecting at least one of proteins or analytes, wherein the type of dye is at least one of an Ab-conjugate, a secondary Ab conjugate, or a strong dye to detect a low abundance analyte.

User Interfaces, as discussed herein, can include an instrument console, a web tool, a graphical user interface, and a display on a computing device.

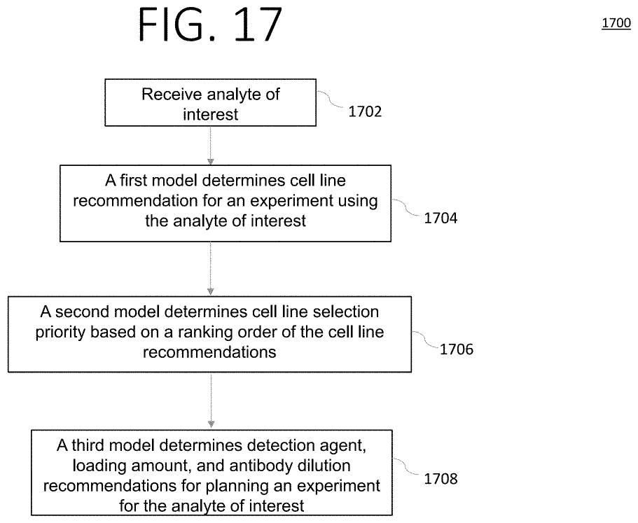

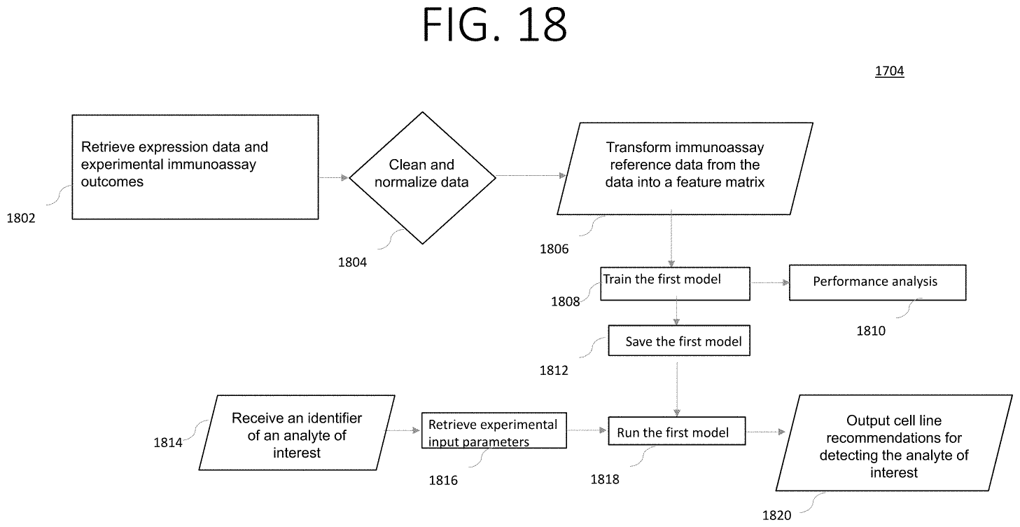

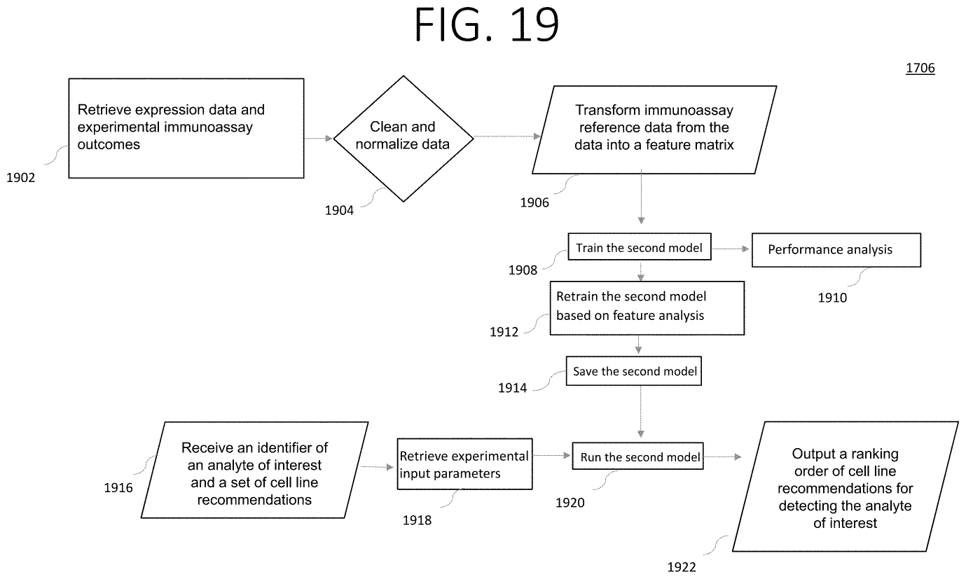

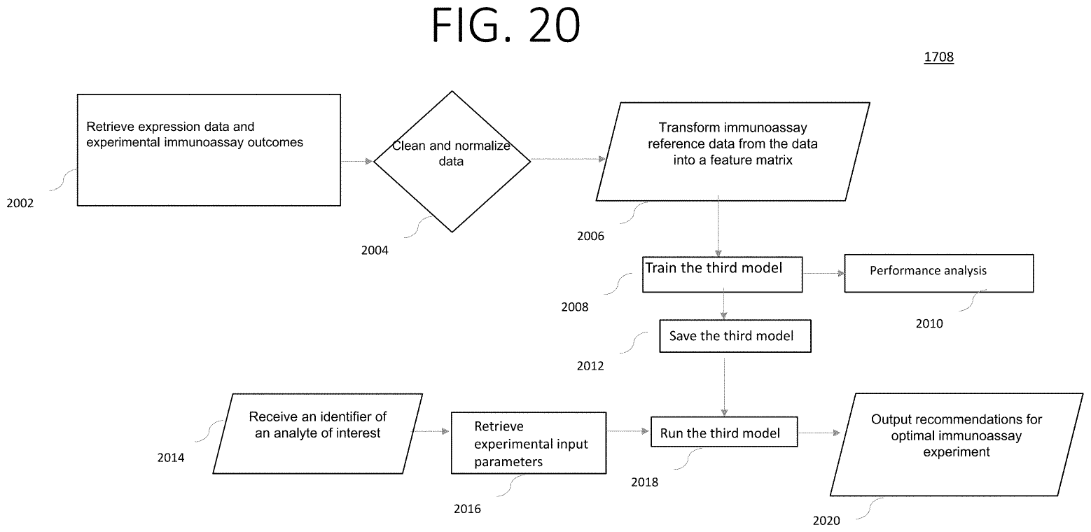

FIG. 17 further illustrates an example method 1700 for identifying target parameters of an experiment based on an analyte using the one or more machine learning networks as described in FIG. 1 and FIG. 2, according to some embodiments. In some embodiments, a plurality of machine learning networks (also referred to herein as “models”) may be used to determine target parameters. At step 1702, an analyte of interest is received. At step 1704, first machine learning model may determine a cell line recommendation for an experiment using the analyte of interest. In some aspects, the first machine learning model may determine a tissue recommendation. Step 1704 is further described by FIG. 18. At step 1706, a second machine learning model may rank the cell line recommendations to determine a selection priority of the cell lines. In some aspects, the second machine learning model may rank tissue recommendations. Step 1706 is further described by FIG. 19. At step 1708, a third machine learning model determines detection agent, loading amount, and antibody dilution recommendations for planning an experiment to be performed on the analyte of interest. Step 1708 is further described by FIG. 20.

FIG. 18 illustrates a method for determining cell line recommendations for use in an immunoassay experiment using a machine learning model. In one aspect, this first model may be trained to identify target parameters based on the analyte. The target parameters may be a cell line or tissue.

At step 1802, expression data may be retrieved from one or more datasets, such as a public RNA-seq curated sources (e.g., ARCHS, CCLE, and other public datasets), and a dataset of immunoassay experimental outcomes may be retrieved. In some aspects, the immunoassay experimental outcomes dataset includes the outcomes of a plurality of immunoassay experiments, where each experiment was performed on an analyte from a plurality of analytes. Each immunoassay experiment outcome may include an analyte, an analyte abundance, a clonality, and a backbone of an antibody.

At step 1804, the expression data and immunoassay experimental outcomes may be cleaned and normalized to prepare the data as immunoassay reference data that may be used in training the first model. At step 1806, the immunoassay reference data may be transformed into a feature matrix. In some aspects, the immunoassay reference data may include reference input parameters and reference analyte detection data. In some aspects, the reference input parameters may include the transcriptomic (TPM) abundance, clonality, and/or an antibody backbone. In some aspects, the antibody backbone may be from one or more mammals, such as a mouse, a rabbit, or a mouse and a rabbit. In some aspects, the reference analyte detection data may identify whether or not the analyte was detected in the cell line or tissue used in each respective immunoassay experiment.

At step 1808, the first model may be trained using the feature matrix. The first model may be a machine learning model. In some aspects, the first model may be a logistic regression model. At step 1810, a performance analysis may be performed on the first model. The performance analysis may be performed, for example, with a validation set of experimental input parameters and corresponding reference analyte detection data. Based on the results of the performance analysis, at step 1812, the trained first model may be saved such that it may be accessed and used by a computing device. Once the first model is saved, it may be used to recommend one or more cell lines or tissues in which an analyte of interest is likely to be detected.

Step 1814 begins the process of using the trained first model to recommend one or more cell lines or tissues in which an analyte of interest is likely to be detected. At step 1814, an identifier of an analyte of interest for an immunoassay experiment may be received. At step 1816, the computing system may retrieve experimental input parameters for the received identifier from a database of analyte data such as, but not limited to, Uniprot, ARCHS, and/or CCLE. The experimental input parameters may be, for example, TPM abundance, clonality, and an antibody backbone such as mouse and/or rabbit antibody backbones. At step 1818, the first model may be run on the experimental input parameters. At step 1820, the first model may output cell line or tissue recommendations for detecting the analyte of interest in an immunoassay experiment. The output may be a list of cell line or tissue recommendations or may be visually presented by a UI, as discussed herein.

FIG. 19 illustrates a method for ranking cell line recommendations for use in an immunoassay experiment using a machine learning model, according to some embodiments. In one aspect, this second machine learning model may be trained to identify target parameters based on the analyte. The target parameters may be a ranking order of cell line or tissue recommendations for an immunoassay experiment.

At step 1902, expression data may be retrieved from one or more data sets, such as public RNA-seq curated sources (e.g., ARCHS, CCLE, and other datasets with literature and/or tested antibodies data), and a target specific feature may be retrieved from one or more data sets, such as public analyte databases such as, but not limited to Uniprot and/or literature tested antibody data. The output obtained from the first model is also retrieved. In some aspects, the target specific features include data for a plurality of analytes. The target specific features may include one or more of the isoelectric point (pI), transmembrane (TM) domain, TPM abundance, turnover, peptide count, and localization.

At step 1904, the expression data, target specific features, and output from the first model may be cleaned and normalized to prepare the data into immunoassay reference data that may be used in training the second model. At step 1906, the immunoassay reference data may be transformed into a feature matrix. In some aspects, the immunoassay reference data may include reference input parameters and reference analyte detection data. In some aspects, the reference input parameters may include one or more of an isoelectric point (pI), transmembrane (TM) domain, TPM abundance, turnover, peptide count, and localization. In some aspects, the reference analyte detection data may be a ranking of the cell line or tissue for detecting the corresponding analyte in an immunoassay experiment.

At step 1908, the second model may be trained using the feature matrix. The second model may be a machine learning model. In some aspects, the second model may be a random forest classifier model. At step 1910, a performance analysis may be performed on the second model. The performance analysis may be performed with a validation set of experimental input parameters and corresponding reference analyte detection data. At step 1912, the second model may be retrained based on analysis of each feature's performance. At step 1914, the second model may be saved such that it may be accessed and used by a computing device. Once the second model is saved, it may be used to rank one or more cell lines or tissues in which an analyte of interest may be detected.

At step 1916, an identifier of an analyte of interest for an immunoassay experiment and a set of cell lines or tissues that are recommended for use in an immunoassay experiment may be received. In some aspects, the set of cell lines or tissues may be obtained from the first model, such as the output of step 1820 in FIG. 18. At step 1918, the computing system may retrieve experimental input parameters for the received identifier from a database of analyte data such as, but not limited to Uniprot, ARCHS, and/or CCLE. The experimental input parameters may include an isoelectric point (pI), transmembrane (TM) domain, TPM abundance, turnover, peptide count, and localization for the analyte of interest. At step 1920, the second model may be run on the experimental input parameters. At step 1922, the second model may output a ranking order of cell line or tissue recommendations for detecting the analyte of interest in an immunoassay experiment. In some aspects, the ranking order may rank the cell lines based on the likelihood that the analyte will be detected in an immunoassay using the cell line. The ranking may order the cell lines while simultaneously ranking the likelihood as being high, medium, or low. In some aspects, the output may be a list of cell line or tissue recommendations or may be visually presented by a UI, as discussed herein.

FIG. 20 illustrates a method for determining cell line recommendations for use in an immunoassay experiment using a machine learning model, according to some embodiments. In one aspect, this third machine learning model may be trained to identify target parameters based on the analyte. The target parameters may be parameters for an optimal immunoassay experiment of an analyte.

At step 2002, expression data may be retrieved from one or more data sets, such as public RNA-seq curated sources such as, but not limited to ARCHS, CCLE, and other public datasets. A dataset of experimental detection data from immunoassay experiments may also be retrieved. In some aspects, the experimental detection dataset includes the outcomes of a plurality of immunoassay experiments, where each experiment was performed on an analyte from a plurality of analytes. The experimental detection dataset may include, for example, one or more of the pixel count, clonality, dilution factor, loading amount, exposure time, and detection agent for each immunoassay experiment.

At step 2004, the expression data and experimental detection dataset may be cleaned and normalized to prepare the data into immunoassay reference data that may be used in training the third model. At step 2006, the immunoassay reference data may be transformed into a feature matrix. In some aspects, the immunoassay reference data may include one or more of the pixel count, clonality, dilution factor, loading amount, exposure time, and detection agent for a plurality of immunoassay experiments.

At step 2008, the third model may be trained using the feature matrix. The third model may be a machine learning model. In some aspects, the third model may be a multivariate linear regression model. In some aspects, the third model may use a plurality of linear regression models, for example Atto, Pico, Dura, and ECL. The third model may use 1, 2, 3, 4, 5, or any other number of models. At step 2010, a performance analysis may be performed on the third model. The performance analysis may be performed with a validation set of experimental input parameters. Based on the results of the performance analysis, at step 2012, the third model may be saved such that it may be accessed and used by a computing device. Once the third model is saved, it may be used to recommend experimental parameters for an immunoassay experiment on an analyte of interest.

At step 2014, an identifier of an analyte of interest for an immunoassay experiment may be received. At step 2016, the computing system may retrieve experimental input parameters for the received identifier from a database of analyte data such as, but not limited to Uniprot, ARCHS, and/or CCLE. The experimental input parameters may be one or more of loading fraction, exposure time, clonality, and dilution factor. At step 2018, the third model may be run on the experimental input parameters. At step 2020, the third model may output a set of recommendations for the optimal immunoassay experiment. For example, the third model may output one or more of the recommended detection agent, loading amount, and antibody dilution for an immunoassay experiment for the analyte of interest. The output may be a list of recommendations or may be visually presented by a UI, as discussed herein.

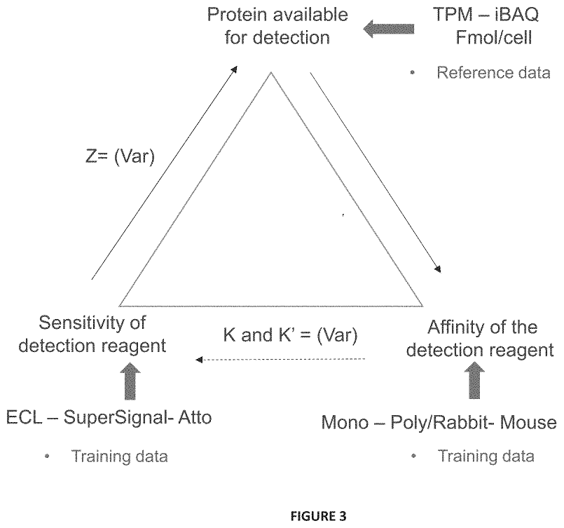

FIG. 3 illustrates relationships between protein, detection agent affinity, and detection reagent sensitivity, in accordance with embodiments described herein. For immunoassay experiments, numerous factors can affect output and successful observations. Balancing the variables and input factors for immunoassay experiments becomes a consideration for effective data collection and analysis.

FIG. 3 highlights three key factors that affect performance data, and which embodiments of the disclosed technology account for in its optimization and recommendation techniques, such as identifying target parameters and immunoassay parameter sets. The available amount of protein for detection, the detection reagent affinity, and detection reagent affinity each affect the efficacy of the other variables. The available protein for detection can be provided via reference data, as discussed herein, for experiments. Transcripts per million/protein intensity-based absolute quantitation (TPM/iBAQ) measured in Fmol/cell is but one example of reference data applicable to available protein for detection.

The affinity of the detection reagent can be obtained and/or provided by training data, e.g., training data for machine learning operations. The affinity of the detection agent can depend, for example, on clonality, e.g., monoclonal or polyclonal, and can be provided using one or more organisms, such as a rabbit or a mouse for example. K and K′ can be indicative of variance, which is related to the sensitivity of a detection reagent. Detection reagent sensitivity can also be obtained and/or provided by training data, and can include, for example, ECL, Pico Plus, SuperSignal, and Atto. Detection reagent sensitivity can consequently affect, and be affected by the available protein for detection, and the relationship between the two can be characterized, in examples, by Z=(Var).

Additional factors which could affect optimal immunoassay data include, but are not limited to secondary antibody effects, membrane effects, blocking effects, protein lability, and extraction effects. For example, unstable proteins can result in a low extraction quality. Data cleansing filters can be applied to data points to exclude outlier effects as well.

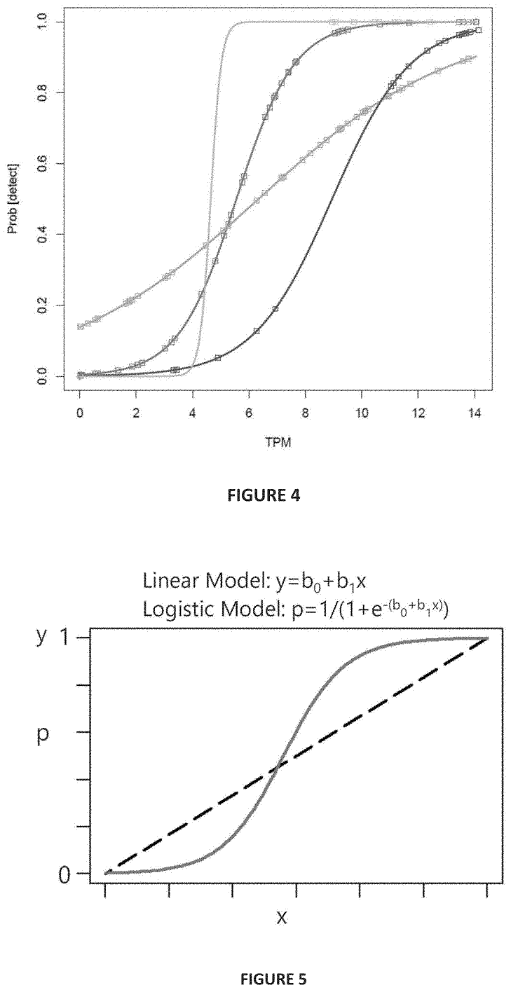

FIG. 4 and FIG. 5 illustrate graphical representations of conceptual frameworks for immunoassays. The figures illustrate that a dose-response curve can be modeled as a sigmoid function. FIG. 4 illustrates example dose-responses for antibody clonality and TPM values. FIG. 5 illustrates a sigmoidal dose-response curve overlaid with a corresponding linear model. The Linear Model of the dose response can be represented by a standard linear formula, y=b₀+b₁x, and the logistic model can be represented by p=1/(1+e\hat{ }−(b₀+b₁x)). These linear and logistic models can be implemented in one or more machine learning algorithms as discussed herein.

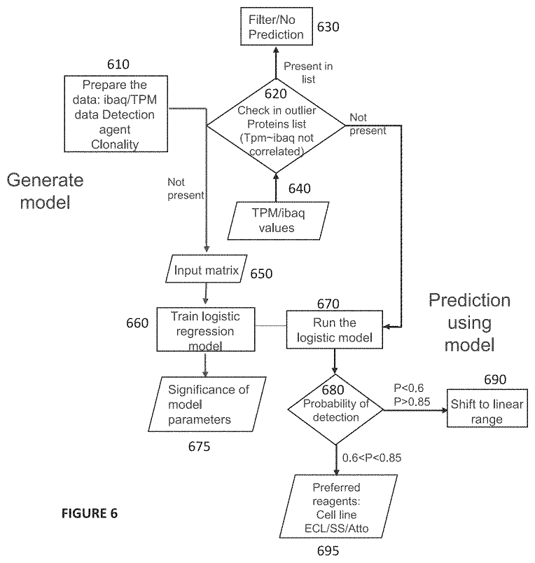

FIG. 6 illustrates an example flow chart for generating a model and providing immunoassay parameter sets, target parameters, and other recommendations and predictions, in accordance with embodiments discussed herein. To generate a model, data preparation 610 can include input on a data detection agent, such as TPM/iBAQ, clonality information, and the like. A query 640 for TPM/iBAQ values can occur in conjunction with a check 620 of an outlier proteins list. In examples, TPM and iBAQ need not be correlated. If the check is positive, identifying that the outlier proteins are present, a filter/number prediction can be output in 630. If the check is negative, indicating that the outlier proteins and correlation are not present, preparation 650 of the input matrix and operation 670 of the logistic model occur.

The input matrix 650 can aid in training 660 the logistic regression model, and utilize information from logistic model run in 670. Based on the logistic regression model training 660, model parameter significance can be output in 675. In addition, the logistic model run in 670 can aid in determining a probability of detection in 680. The detection probability assists in determining preferred reagents and/or a possible shift to linear ranges. In an example, if the probability is 0.6<P<0.85, then recommended preferred agents 695, such as cell line ECL/SS/Atto, are provided. In cases where P<0.6 or P>0.85, the model prediction shifts to a linear range in 690. Endpoints 0.6, 0.85 can optionally be associated with either option, and in examples, can be defined and/or chosen by a user.

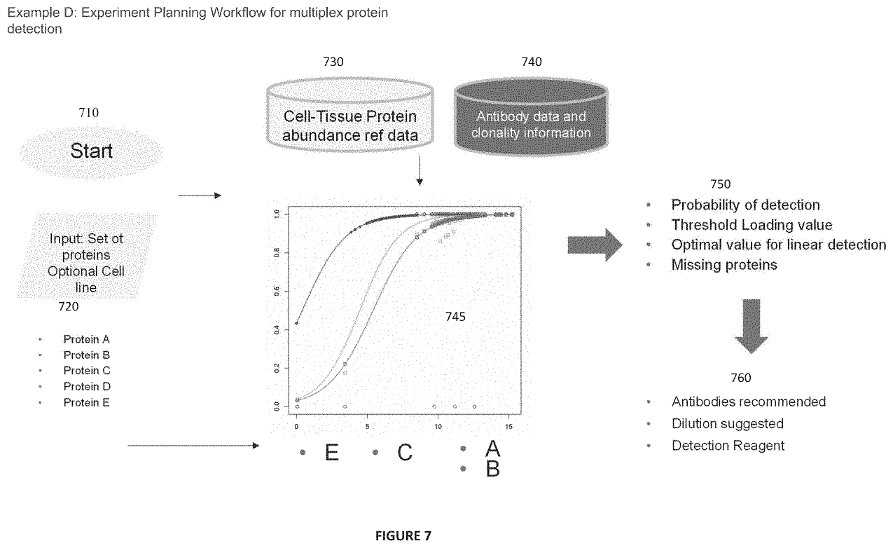

FIG. 7 illustrates an example experiment planning workflow for multiplex protein detection. The experiment can first utilize a set of proteins and an optional cell line for input in 720. In the present example, the set of proteins can comprise five proteins (A-E). A plurality of experimental data and reference data can provide additional information for the detection recommendation and probability determinations. In examples, cell-tissue and protein abundance reference data 730 and/or antibody data and clonality information 740 comprise reference data for models discussed herein. Embodiments can generate a graphical representation 745 of predicted output for each of the proteins A-E. The graphical representation and associated data can provide recommendations 750 and predictions 760, for one or more experimental variables and/or outputs.

In examples, predictions and recommendations can comprise immunoassay parameter sets, a probability of detection, a threshold loading value, a target value for linear detection, and missing proteins. Moreover, output can provide recommended antibodies, a suggested dilution for one or more products, and a recommended detection agent.

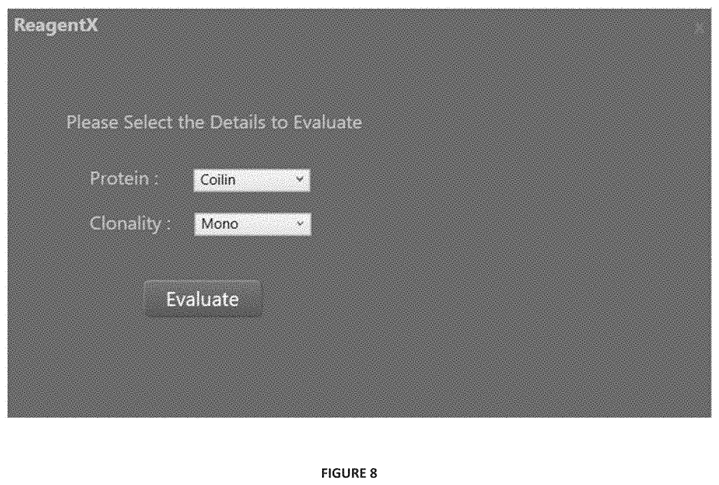

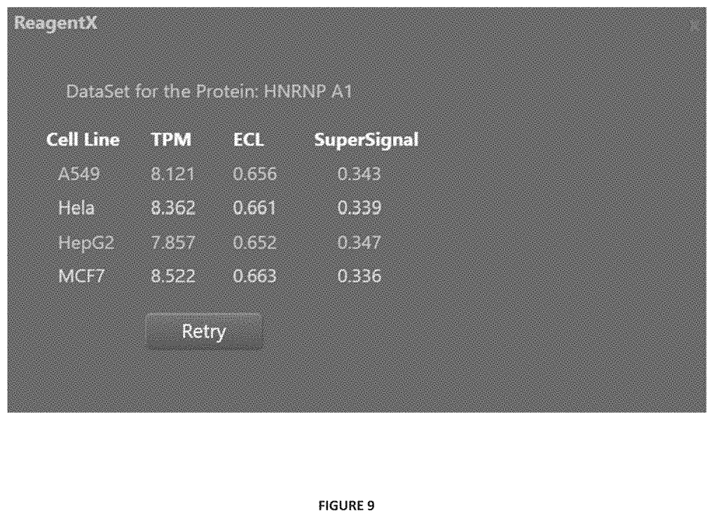

FIGS. 8-9 illustrate user interfaces associated with embodiments discussed herein. Embodiments can be provided on one or more computing systems, displays, web tools, applications, and the like. FIG. 8 illustrates an initial screen, wherein a user has the option to select experimental details to evaluate. In the depicted example, a user can select a type of protein (e.g., coilin), and a clonality (e.g., mono, poly). It will be appreciated that the details provided on the user interface need not be limited to the depicted categories, and can be adjusted based on any of a plurality of factors, including but not limited to an experiment type, a desired outcome, typical variables, particular variables of interest, and the like.

The UI may be utilized to perform the method as described by FIG. 1. In one aspect, the UI can facilitate the planning of an experiment, specifically a Western blot experiment, such that the experiment performs optimally with little to no testing. The UI can be hosted by a host server and can be connected to the Internet. In other aspects, the UI may not be connected to the Internet but may instead reflect operations performed locally.

In one aspect, the UI may prompt a user to plan a new experiment. When a user selects to plan a new experiment, the module will provide recommendations for reagents that may be used to detect an analyte of interest, such as a protein of interest. In other aspects, implementations of the UI may be used to provide recommendations for a variety of analytes as described herein.

FIG. 9 illustrates an example dataset output for a selected protein, e.g., which may be input on an initial screen like that of FIG. 8. The dataset output can provide information related to the use of one or more of a cell line, TPM, ECL, and SuperSignal. As such, the output can inform users on one or more output predictions, which can be used to aid experimental design and identify target variables and conditions for the desired experiment.

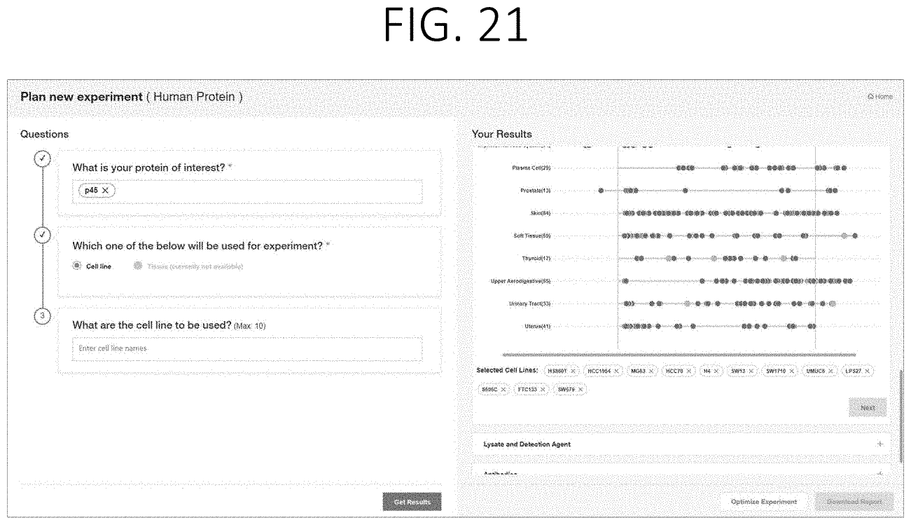

FIG. 21 shows an alternate version of FIG. 8. As shown in FIG. 21, the UI may prompt the user to input a protein. The input may be selected from a menu or may be entered in free text. The protein can be input using, for example, its name, Uniprot ID, alternative name, gene name, and/or gene synonyms. The UI may show a list of suggestions for the protein as a look-up table and the user may select the protein from the table. In some aspects, the user may input multiple proteins of interest.

When the UI receives the protein, it may prompt the user to select whether a cell line will be used to complete the experiment. In some aspects, the user may select that a tissue be used for the experiment instead of a cell line.

The UI may also prompt a user to optionally input a selection of cell lines. If the selection of cell lines are input, the model may determine if the protein may be detected from any of the cell lines in the selection of cell lines. If no cell lines are input, the model will determine if the protein may be detected from any of the cell lines in a complete list of available cell lines.

The user may select to receive results for the protein. The results may include one or more of data for the protein's annotation, cell lines, detection reagent, and antibodies.

The protein annotation results may display information for the protein. The information displayed may include, but is not limited to, the protein's name, Uniprot ID, gene name, mass, post-translational modification, and/or isoelectric point (pI).

As illustrated in FIG. 21, the cell line results may provide recommendations for the cell lines in which the protein is most likely to be detected when performing the experiment. The cell line results may report the determined optimal cell lines that are obtained from the first model, as described by FIG. 18. The UI may display the cell line recommendations with a pictorial representation of the cell lines categorized by lineage and probabilities of detection, as illustrated, for example, in FIG. 21. Each cell line in the pictorial representation may be depicted by a mark, for example a dot. The user may be able to hover over each mark to display the cell line. In some aspects, the cell culture conditions may also be displayed when hovering over the mark.

If the user inputs multiple proteins, the cell line results may display the recommendations for cell lines in a matrix with cell lines on one axis and the proteins on the other. The matrix will display “yes” or “no” depending on whether the protein can be detected in the corresponding cell line. In some aspects, a user can hover over the cell line and cell culture conditions and lineage of the cell line may be displayed.

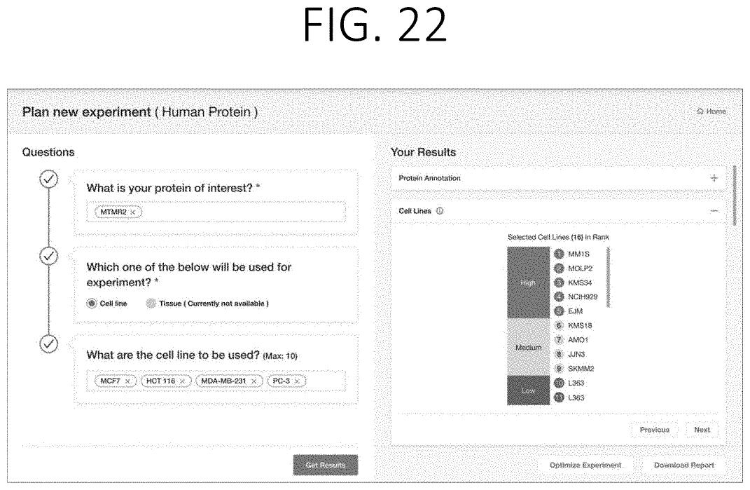

As illustrated in FIG. 22, after receiving the cell line results, a user can select cell lines from the displayed cell lines. The selection of cell lines may be input into the second model, which will rank the selected cell lines based on the protein's likelihood of being detected in the cell line as described by FIG. 19. In some aspects, the UI may display the ranking and categorize the cell lines as “high”, “medium”, or “low” in regards to potential detection.

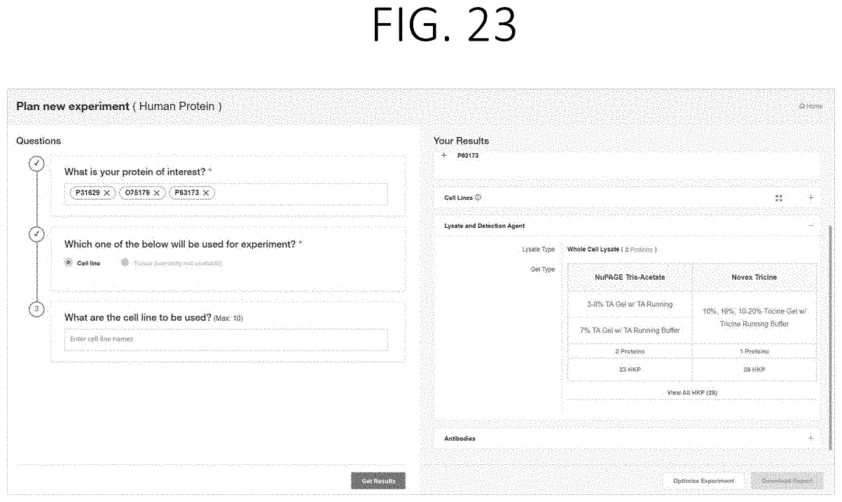

As illustrated in FIG. 23, an example lysate and detection reagent result may display experiment recommendations for lysate type, gel type, and house-keeping proteins (HKP) to be used for the protein. The recommendations may be determined from a data lookup model that utilizes known protein masses to match with recommended lysate types, gel types, and HKP for the protein mass. In some aspects, the lysate type may be displayed with the number of proteins for which it can be used. In some aspects, the gel types are displayed with the proteins that can be detected using the gel type. In some aspects, the HKP may be displayed as the number of recommended HKP, for example, 23 recommended HKP may be displayed as “23 HKP”. A user may select the numbered HKP recommendation and the UI may display the list of recommended HKP with each HKP's name, mass, clonality, and/or backbone, along with the corresponding SKU link for the antibodies which may be used to detect the HKP.

Similarly, an example antibodies result may be displayed with its name and SKU link. In some aspects, a link can be available on the results page that may direct a user to a webpage that details all available primary antibodies.

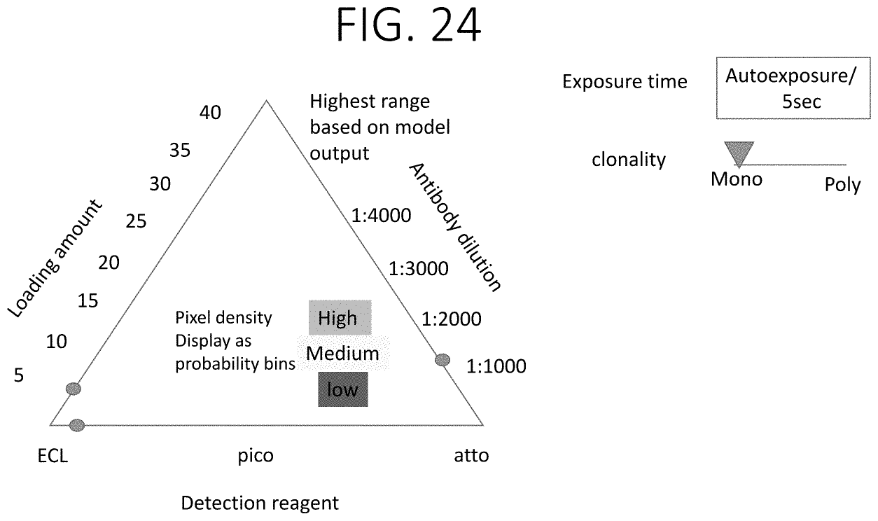

As illustrated in FIG. 24, the UI may display one or more recommendations for planning an experiment with the protein. The UI may visually display the results from the third model as described by FIG. 21. These results include the recommended loading amount, antibody dilution, and detection reagent for detecting the protein of interest. In some aspects, the pixel density for the experiment may be displayed as being “high”, “medium”, or “low”.

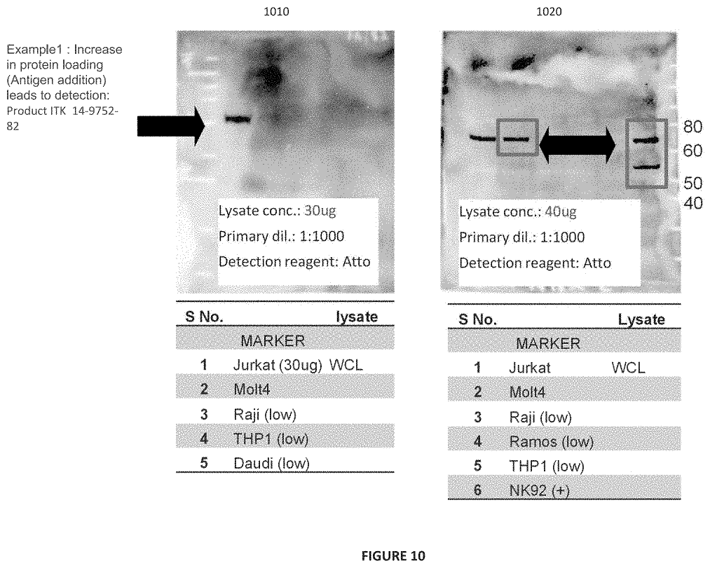

FIG. 10 illustrates an example scenario related to parameter optimization and recommendation. In the illustrated example, experiment 1010 and experiment 1020 illustrate an output difference related to an increase in protein loading. In experiment 1010, the lysate concentration is 30 μg, and experiment 1020 increases the lysate concentration to 40 μg. In both experiments, the primary dilution is 1:1000 and detection reagent is Atto. As such, the difference in results can be attributed to the lysate concentration difference.

In this example and other common user scenarios, a low detection result (i.e., experiment 1010) can often be attributed to one or more of: low protein abundance, a loss in extraction, a lower antibody affinity base, excess blocking, and a weaker detection agent. In such scenarios, low detection can be addressed by one or more of the following actions: loading additional protein, reducing blocking, revising protein preparation, changing a membrane type, using high sensitivity detection agents, and replacing the antibody. Since each of the above factors can affect the experiment slightly differently, and a user must decide which factors to adjust and test, the number of trials/experiments that a user implements increases drastically. For example, if there are 6 factors that affect detection, and 3-4 possible user adjustments per factor, that results in 24-30 user trials to test out factors and identify those that optimize detection. Accordingly, applying the machine learning models discussed herein can efficiently test and predict experimental outcomes for a plurality of factors, and recommend ideal factors for experimental use.

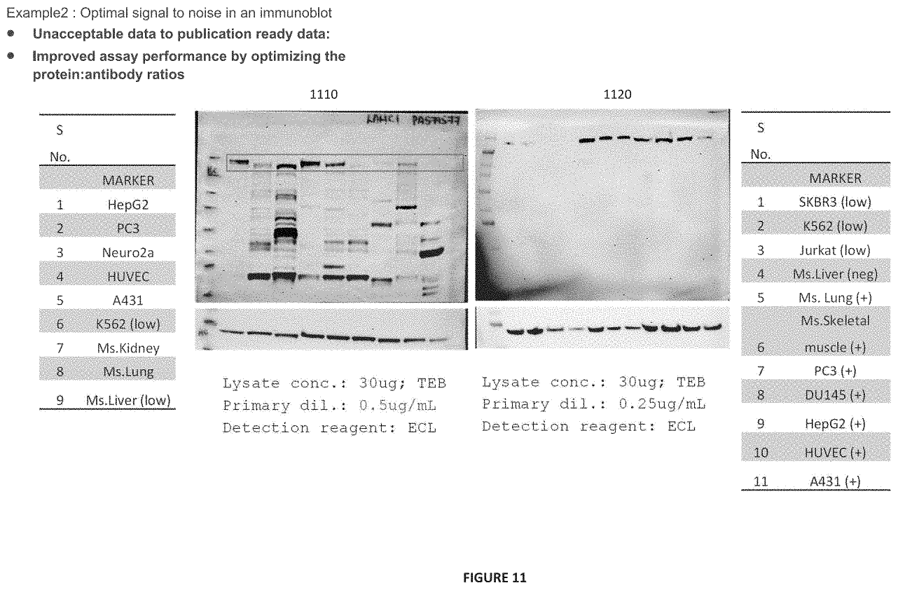

FIG. 11 illustrates another common user scenario, wherein a target signal to noise ratio requires identification. In various examples, optimizing the signal to noise ratio can improve data from being unacceptably noisy to publication-ready. Moreover, such improvements can improve assay performance by identifying a target protein to antibody ratio. In the illustrated experiments of FIG. 11, experiment 1110 utilizes a primary dilution of 0.5 μg/ml, and experiment 1120 utilizes a primary dilution of 0.25 μg/ml. The lysate concentration and detection reagent remained the same between experiments. As a result of the dilutional change, experiment 1120 resulted in a cleaner immunoassay, where markers can be clearly identified.

In these scenarios, a poor signal to noise ratio can result from a plurality of factors, including but not limited to a non-optimal protein load, a degraded preparation, a poor antibody volume, an excess antibody volume, and sub-optimal blocking. User responses to address those issues often include one or more of: an increase in protein load, an increase blocking, protein preparation revision, changing the membrane type, diluting the antibody, and replacing the antibody. The machine learning models discussed herein can assist in testing those factors and predicting, based on prior data and model input information, experimental recommendations.

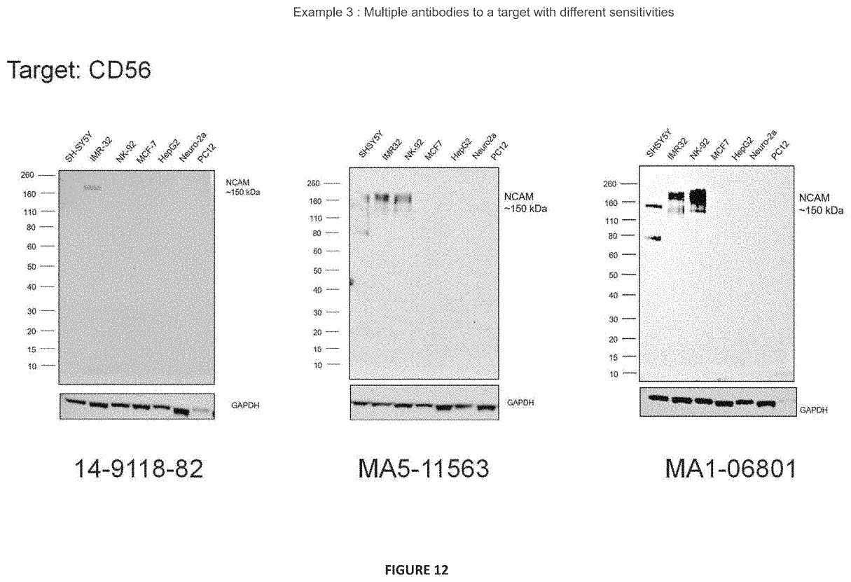

FIG. 12 illustrates a set of experiments utilizing multiple antibodies with different sensitivities. FIG. 12 illustrates how experimental results can change drastically, based on the sensitivity levels. Such information, including the experimental data from each example, can provide reference data useful for training one or more machine learning models in accordance with embodiments discussed herein. The depicted experiments demonstrate the effect of sensitivity differences, which can provide useful information for experiments utilizing similar antibodies and targets.

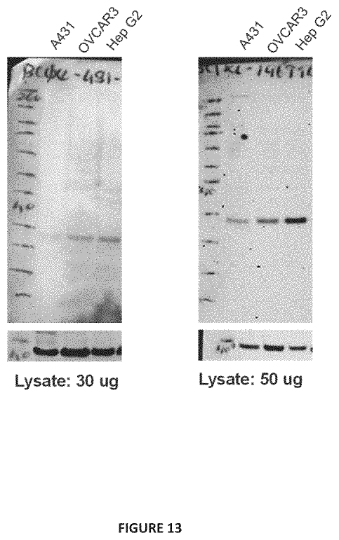

FIG. 13 illustrates another set of experiments, with differing results upon changes in a variable. In the depicted example, an increase in lysate load from 30 μg to 50 μg provides the necessary amount for detection. Again, such data can provide helpful information for machine learning models and predicting effects on lysate load increases.

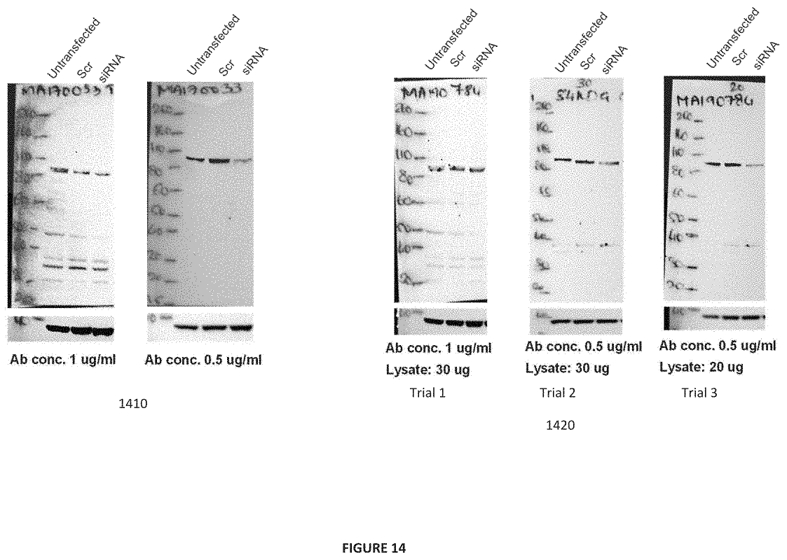

FIG. 14 illustrates another example experiment. In this scenario, reducing protein concentration can improve protein quantification. Antibody titration, can be implemented in examples, to assist with adjusting the antibody concentration, and ultimately protein quantification. Experiments 1410 and 1420 illustrate improved effects from a decrease in antibody concentration. In experiment 1410, the Ab concentration decreases from 1 μg/ml to 0.5 μg/ml, and detection clarity improves. In experiment 1420, the Ab concentration decreases from 1 μg/ml to 0.5 μg/ml between the first and last two trials, and the lysate concentration remains at 30 μg for the first two trial sand decreases to 20 μg for the last trial. The initial decrease in Ab concentration between Trials 1 and 2 provide a sharpened detection result, and the subsequent decrease in lysate concentration between Trials 2 and 3 further clarify the detection result. As with the prior experiments discussed herein, the experimental results provided in FIG. 14 can provide reference data for machine learning models, and aid in experimental predictions and recommendations.

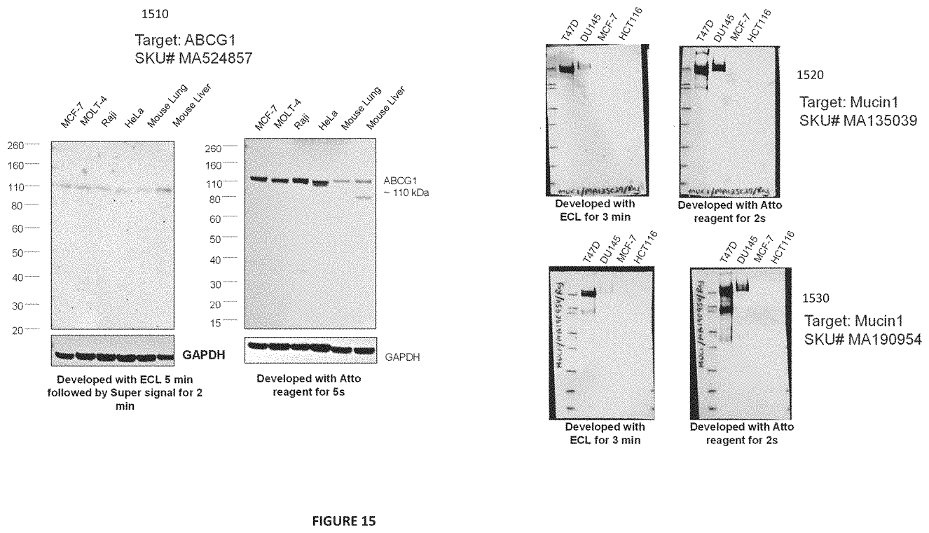

FIG. 15 illustrates experimental differences that can occur with enhanced reagent sensitivity. In experiment 1510, a first trial is developed with ECL for 5 minutes followed by SuperSignal for 2 minutes. The second trial is developed with Atto reagent for 5 seconds. The second trial resulted in clear detection results. In experiments 1520, 1530, a first trial was developed with ECL for 3 minutes, and a second trial was developed with Atto reagent for 2 seconds. In both cases, the change in reagent increases detection.

### Troubleshooting

It is expected that an analyte will migrate through a gel (or other membrane depending on the type of electrophoresis used) based on a function of mass (molecular weight), so that analytes of different mass can be identified as separate bands within an immunoblot. Such techniques may be used, for example, to identify and quantitate proteins during protein preparation. However, there are many factors that may cause an analyte to not migrate as expected. For example, the charge of the analyte, post-translational modifications (e.g., glycosylation, lipidation), gel type, and buffer type may all affect the analyte's migration. This difference in migration is referred to herein as a “shift”—a physical shift between an observed migration and an actual (that is, expected) molecular weight for a given analyte. This shift may affect the results of the immunoassay, leading to errors in or misleading immunoassay results.

It is therefore necessary to develop a method that may determine the migration shift of an analyte in an immunoassay such that the results of the immunoassay are still accurate and usable. Because there are multiple factors that affect migration, and some of those factors are not linear, linear calculations may not be effective in determining the shift. Therefore, in accordance with aspects described herein, a neural network may be used in order to determine the degree an analyte has migrated or shifted in an immunoassay experiment.

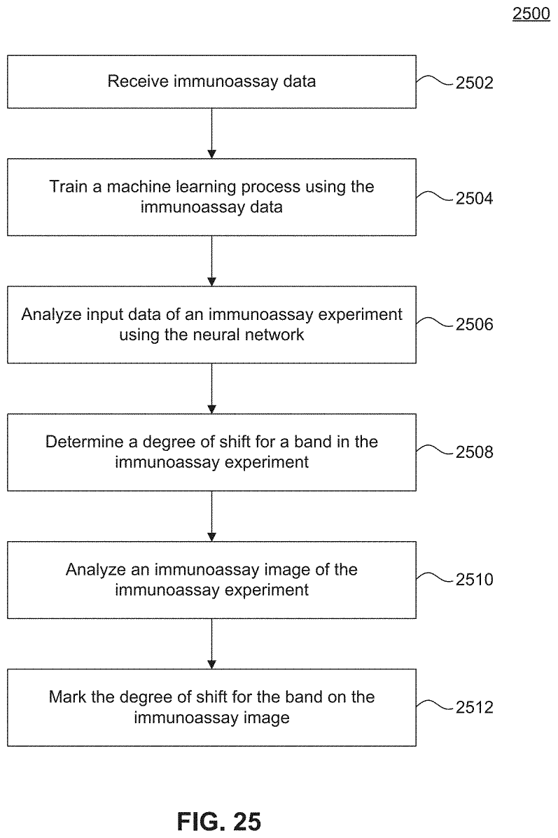

FIG. 25 shows a method 2500, according to some aspects. For example, method 2500 may be used to determine the shift of an analyte in an immunoassay. Analytes are specimens extracted for analysis from a portion or sample using a specific extraction protocol and typically a substance of interest that needs detection. In many cases the analyte is a protein, although it may be other kinds of molecules, of different sizes and types, as long as a method for tagging the analyte is available (e.g. staining or antibody detection). In some aspects, the analyte described herein may be a protein, hapten, hormone, nucleic acid, peptide, modified peptide, or a modified form of any of the foregoing analytes.

At step 2502, a computer system may receive an immunoassay dataset with known information for a plurality of known analytes. The immunoassay dataset may be received from a plurality of sources, including but not limited to a database of analyte data, a researcher, or an organization. The immunoassay dataset may include data that was collected from immunoassay experiments of the plurality of analytes. In some aspects, the immunoassay experiments may be a bead-based immunoassay, for example, a multiplex assay, a bead-based immunoassay utilizing panels, or a bead-based immunoassay utilizing activated surfaces panel builder. In other aspects, a flow-based immunoassay can be a lateral flow immunoassay, a flow assay that uses colored particles, a competitive assay, and the like. In some aspects, the immunoassay may be a single or multiplex Western blot, or an SDS-PAGE (Sodium Dodecyl Sulfate-Polyacrylamide Gel Electrophoresis) gel.

The immunoassay dataset may comprise sets of input parameters, which can correspond to an analyte's degree of shift in a gel. In some aspects, the sets of input parameters may include a variety of features, including, but not limited to, post-translational modifications such as phosphorylation, glycosylation, ubiquitination, nitrosylation, methylation, acetylation, lipidation and proteolysis, to name a few, interchain or polymer cross-links, disulfide groups, modified residues, isoelectric point (pI), gel type, and buffer type, that each correspond to an immunoassay for a specific analyte. Each set of input parameters corresponds to a degree of shift for an analyte. The immunoassay dataset may have a variation of reagents, cell lines, analytes, antibodies, gel types, buffer types or any other variation of the foregoing features, from which the different attributes contributing to a molecular weight shifting may be identified by a neural network. In some aspects, the immunoassay dataset may include negative data.

At step 2504, a machine learning process may be used to determine the degree of shift in a band experienced by a particular analyte in an immunoassay. Machine learning involves the development and use of computer systems that are able to learn and adapt without following explicit instructions, by using algorithms and statistical models to analyze and draw inferences from patterns in data. Machine learning models suitable for the disclosed embodiments may include, for example and without limitation, supervised learning, semi-supervised learning, unsupervised learning, or reinforcement models. Supervised learning models may be trained on labeled data, where example conditions associated with a desired output are fed to the machine learning model during training. Some non-limiting examples of supervised learning models include, for example and without limitation, nearest neighbor, naïve Bayes, decision trees, support vector machines, neural networks, or any machine learning algorithm suitable for image analysis and/or ranking problems. In some embodiments, the machine learning process may include a feedforward non-deep neural network or a deep learning network. For the purposes of this disclosure, a non-deep feedforward network will simply be referred to as a neural network. The neural network may be trained using the immunoassay dataset to determine the degree of shift in a band experienced by a particular analyte in an immunoassay. In some aspects, the neural network is developed on a computer system that includes a memory and a processor. The neural network may be built with any neural network architecture, e.g., unsupervised pre-trained networks, convolutional neural networks, recurrent neural networks, recursive neural networks, or the like. In some aspects, a neural network has at least two hidden layers.

The neural network may be trained using the immunoassay dataset, where the input parameters, also known as features, and corresponding analyte's degree of shift may be used as input. In some aspects, the input parameters may include glycosylation of the analyte. In some aspects, the input parameters may include glycosylation, disulfide bonds, modified residue, MOPS (3-(N-morpholino)propanesulfonic acid), and ubiquitination. In some aspects, the input parameters may include glycosylation, disulfide bonds, modified residue, MOPS (3-(N-morpholino)propanesulfonic acid), ubiquitination, lipidation, IVIES (2-(N-morpholino)ethanesulfonic acid), isoelectric point (pI), 4-12% gel type, 10% gel type, SUMOylation, Tris acetate, 3-8% gel type, 12% gel type, and cross links. In some aspects, the input parameters may include additional or fewer parameters than those listed. In some aspects, the input parameters may include polymer modifications, features that affect charge, and the type of gel. The features may be extracted from an existing database (e.g. Uniprot). The neural network may be trained to output an analyte's degree of shift in the gel.

At step 2506, once the neural network is trained, the neural network may analyze experimental data of an analyte of interest for an immunoassay experiment. An immunoassay experiment may be completed for the detection of the analyte of interest and the experimental data may be input into the neural network. The experimental data may include data for the analyte of interest and specific experimental parameters of the immunoassay experiment including, but not limited to, post-translational modifications, glycosylation, lipidation, disulfide groups, modified residues, and isoelectric point for the analyte of interest and gel type and buffer type for the immunoassay experiment.

At step 2508, when the analyzing is completed, a degree of shift for a band comprising an analyte of interest in the immunoassay experiment is determined based on the output of the neural network. The shift is determined by the degree that the band is predicted to shift according to the results from the analysis by the neural network. In some aspects, the band may be observed in the immunoassay experiment and comprises the analyte of interest.

At step 2510, in some aspects, an immunoassay image taken of the immunoassay experiment may be analyzed and marked. The analysis may mark the frames, lanes, and bands of the immunoassay image. The analysis may be performed by an analysis software, e.g., iBright™.

At step 2512, the band's degree of shift may be marked on the immunoassay image and displayed for a user to visually observe the degree that the band of the analyte of interest shifted.

Once the degree of shift is determined, the neural network may link the cause of the degree of shift to one or more of the experimental parameters, based on the immunoassay dataset on which the neural network was trained. The neural network can then be used to identify changes to the experimental parameters in order to optimize the parameters of the experiment for the sample and analyte of interest.

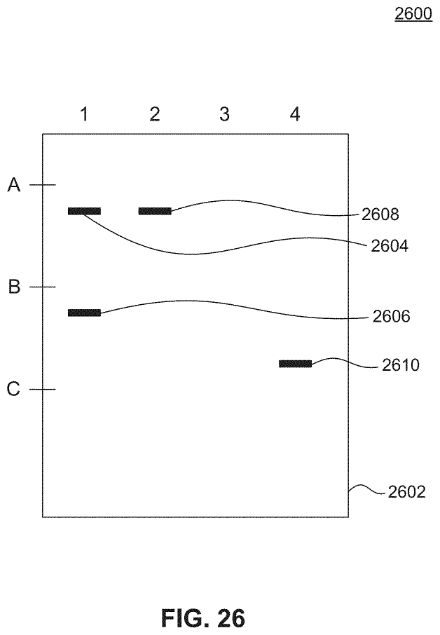

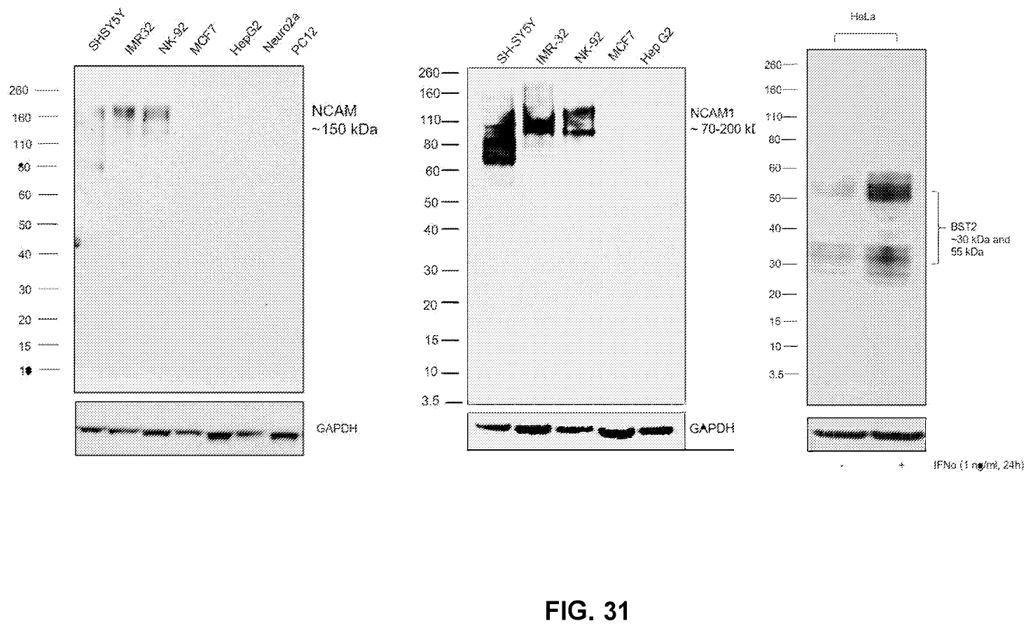

FIG. 26 is an example diagram of an immunoassay experiment 2600. In some aspects, immunoassay experiment 2600 may be a Western blot experiment. Each number on the x-axis signifies a lane and each letter on the y-axis signifies a molecular weight/mass. In this example, a band of interest is a band that is located at the degree of shift that is determined by the neural network in step 2508. Frame 2602 frames the scope of the entire immunoassay experiment. In lane 1, band 2604 is the band of interest. In lane 1, there may also be a band 2606 that is a non-specific band. Non-specific bands are determined to be bands of an analyte that were marked by a detecting reagent but do not include the analyte of interest. Therefore, it would be reported that the band of interest was found with a non-specific band. In lane 2, band 2608 is also a band of interest. Therefore, it would be reported that the band of interest was found in lane 2. Lane 3 has no bands and it would therefore be reported that lane 3 is a blank lane. In lane 4, band 2610 is not where the band of interest was determined to be. It is therefore reported that lane 4 has no band of interest but does include non-specific bands. In certain cases, where the analyte may exhibit post-translational modifications (PTMs) in a natural cellular or tissue context, or under specific treatment conditions, or in a cell cycle stage specific manner, multiplicity of bands may be encountered. In such cases, depending on the number of sites available for PTMs, several bands within a defined range with differential shifts of mass will be reported as bands of interest. The multiplicity of bands may be understood by the example immunoblot shown FIG. 31.

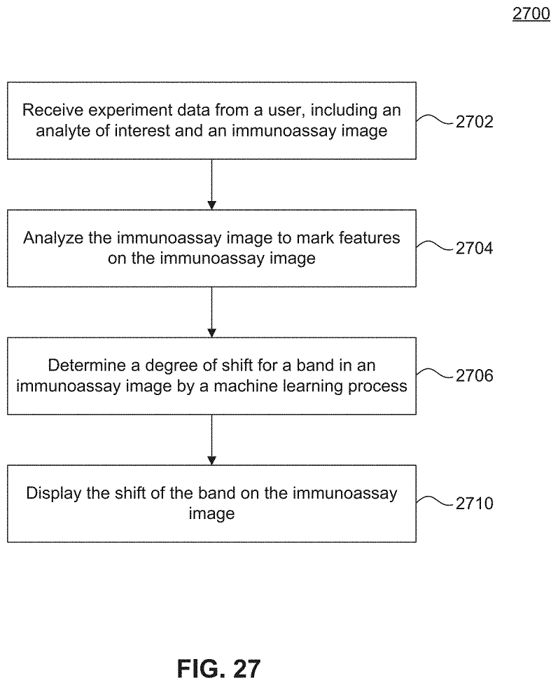

FIG. 27 shows a method 2700, according to some aspects, for a system that may be used to determine the degree of shift of an analyte's band that has occurred. In some aspects, method 2700 may be performed by a computer system that has a memory and a processor. In some aspects, a user interface (UI), e.g., website, application, data/content source, etc., with which a user can interact, may facilitate method 2700 to determine and display a shift in the analyte's band in an immunoassay, such as a western blot experiment, such that the experiment performs optimally. The UI may be hosted by a host server and can be connected to the internet. In other aspects, the UI may not be connected to the internet. In some aspects, the UI may be implemented in an existing software, e.g., iBright™, Thermo Fisher Connect Platform™, etc.

In some aspects, method 2700 may be initiated by a user to troubleshoot an experimental result that is suboptimal. For example, method 2700 may be initiated when a result of an immunoassay analysis does not include or match an expected result, such as when the band detected on the Western blot differs from that expected for the analyte of interest. Method 2700 may also be initiated by a user to quality check the system's accuracy even if it is not suspected that the output is incorrect.

At step 2702, the system may receive experimental data from a user. The experimental data includes an identifier of an analyte of interest and an immunoassay image. In some aspects, the experimental data may also include, for example and without limitation, one or more of an identifier of a cell line, an identifier of a molecular marker, a lysate type, a loading concentration, or a gel type that was used in the immunoassay experiment. In some aspects, the cell line may be a tissue lysate, recombinant protein, or synthetic protein.

The identifier of the analyte may refer to a name of the analyte. The name can be, for example and without limitation, a common name, a scientific or molecular name, a user-defined name, a symbol, a code, or other method of referring to and identifying a type of analyte.

The immunoassay image may be an image of an immunoassay experiment that was performed to detect the analyte that corresponds to the identifier. In some aspects, the immunoassay image may be an image of a bead-based or a flow-based immunoassay experiment. In some aspects, the immunoassay image may be an image of a Western blot experiment. In another aspect, the immunoassay image may be a stained image of an SDS-PAGE gel. The immunoassay image may have features that include a band, a lane, and a frame. A band is the location that an analyte migrates to in the immunoassay. In some aspects, there may be a plurality of bands in one immunoassay image. A lane is a specific panel of the immunoassay image where one experiment is completed. In some aspects, an immunoassay image may have a plurality of lanes when there are a plurality of experiments completed on the same immunoassay. A frame comprises every lane and band in the immunoassay image.

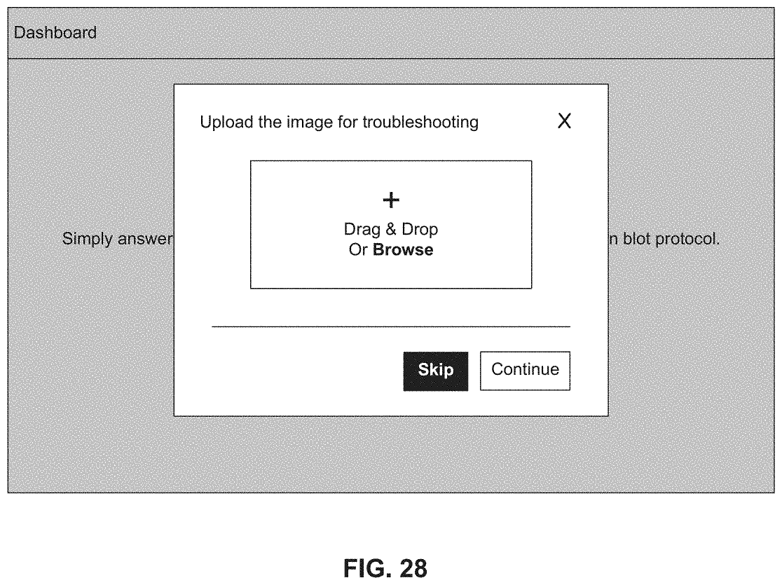

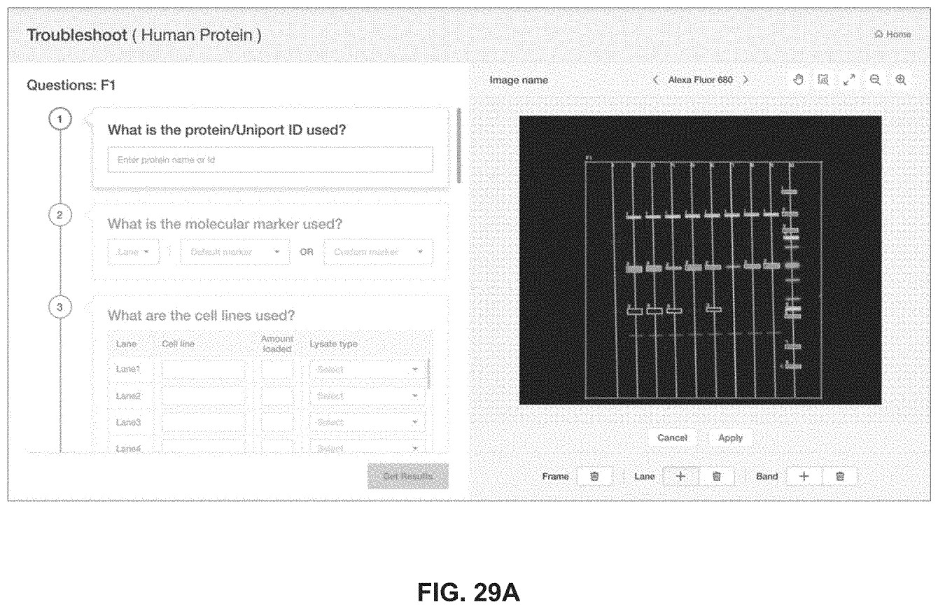

FIG. 28 is an example of a UI that may be used when performing step 2702, according to some aspects. In some aspects, the UI may be included in the UI that utilizes the methods in FIG. 1. A popup window may be displayed that allows a user to browse and select a file, such as an immunoassay image, of the experiment. In some aspects, the immunoassay image may be dragged and dropped into the popup window or the file may be selected from an accessible file listing. Once the user selects the file, the file will upload. Upon completion of the upload, a “troubleshoot” workflow may be launched.

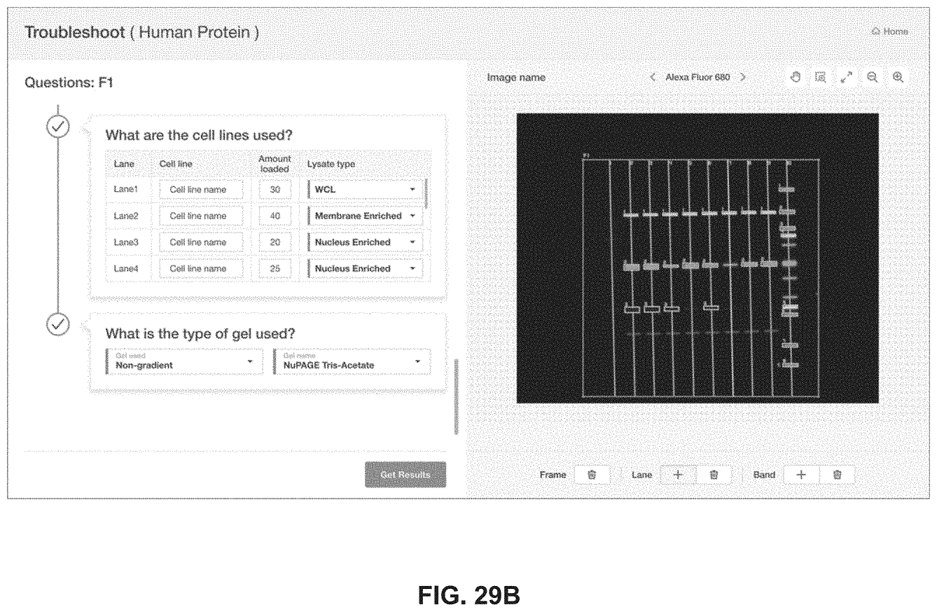

FIGS. 29A and 29B are examples of a UI page that may be displayed after the immunoassay image is uploaded, according to some aspects. The UI may prompt the user to input experimental data about the immunoassay experiment. For example and without limitation, the experimental data may include the identifier for the analyte used, the molecular marker used, the gel type used, the cell lines used in each lane, the loading concentration of each lane, and/or the lysate type used in each lane for the immunoassay experiment. In some aspects, the user may be able to adjust the analysis of the image, such as by editing one or more of the frames, lanes, or bands. In some aspects, the UI may present a series of questions for the user to answer to help guide the troubleshooting process. Questions may be related to, for example and without limitation, the experimental data noted above.

Returning to FIG. 27, at step 2704, the immunoassay image may be analyzed to mark the features in the immunoassay image. This may include any bands, lanes, or frames found in the immunoassay image. In some aspects, the analysis may be performed by an analysis software. In some aspects, the UI may provide a user the ability to adjust the results of the analysis by editing the frames, lanes, and/or bands of the image.

At step 2706, a neural network, as described in step 2504, may be used to determine a degree of shift that has occurred for a band on the immunoassay image. The band may be the band that comprises an analyte of interest corresponding to the identifier that was received in step 2702. The experimental data received from the user in step 2702 may be input into the neural network for analysis. The identifier may be used by the system to retrieve data about the analyte that will be input into the neural network as well. The degree of shift of the band is then determined. If there are a plurality of lanes, each lane may be analyzed separately by the neural network to determine the shift of the band in each lane.

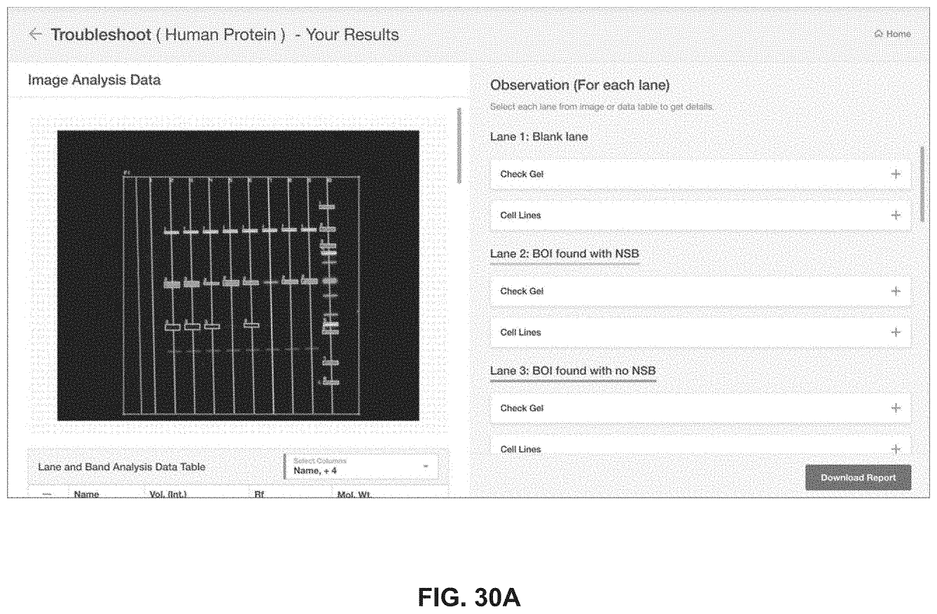

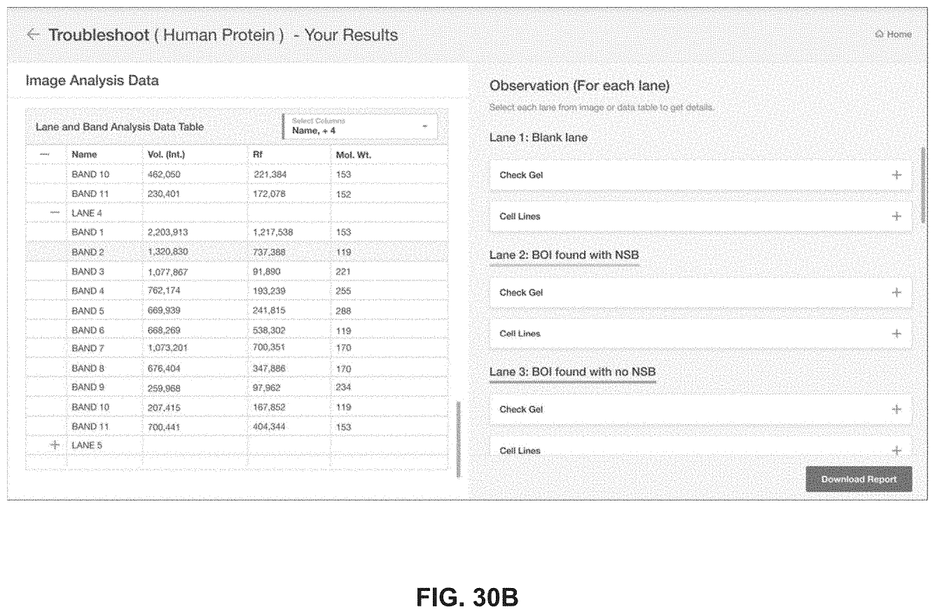

At step 2708, the degree of shift of the band will be displayed on the immunoassay image. In some aspects, the shift may be displayed by the UI. FIGS. 30A and 30B are examples of a UI page that may be displayed when the shift is determined. If the band of interest has not been detected, the UI page may display information about what could have gone wrong in the experiment.

In some aspects, observations for each lane are provided by the UI, based on the results of the neural network. In some aspects, the UI may display the shift of the band for each lane, regardless of whether the band of interest is detected or not. In some aspects, a band of interest may be highlighted in each lane on the immunoassay image. In some aspects, observations of every lane may be displayed to the user via the UI, or a subset of lanes may be displayed to the user based on the receipt of a filtering parameter. Accompanying data and/or details may be provided to the user if there was a problem with the experiment, such as using the gel incorrectly or where the cell line is not suitable for the answers sought. For example, for each lane, a user may see one of the following conclusions: (1) the band of interest was found; (2) the band of interest was found along with non-specific bands; (3) no band of interest was found; and (4) no bands at all were found, such that the lane was blank.

In some aspects, a molecular weight for each band based on the experimental data for the molecular marker is calculated by the computer system. The molecular weights may then be displayed in a data table. FIG. 30B shows an example of a UI page that displays the data table along with observations.

The system may also determine whether and/or how the experimental data should be modified for each lane depending on the results of the immunoassay image and analysis. In some aspects, the modifications may be determined by a relationship between the experimental data and known analyte detection data. In some aspects, the modifications may include suggestions of whether the gel type or cell line used in each lane are suitable for detecting the analyte of interest. In some aspects the UI may display the determined modifications.

In some aspects, a user can select a particular band to get further details regarding the results of the troubleshooting analysis.

Based on the band shift, results, and band conclusions of the experiment, the system may initiate a module that plans an experiment based on the results of the troubleshooting analysis to optimize the immunoassay experiment and provide a plan for detection of the analyte of interest. For example, using a prediction model the plan may suggest differences in the experimental setup, such as the exposure time and/or amount of lysate that needs to be loaded so as to optimize the level of analyte detection in a given range.

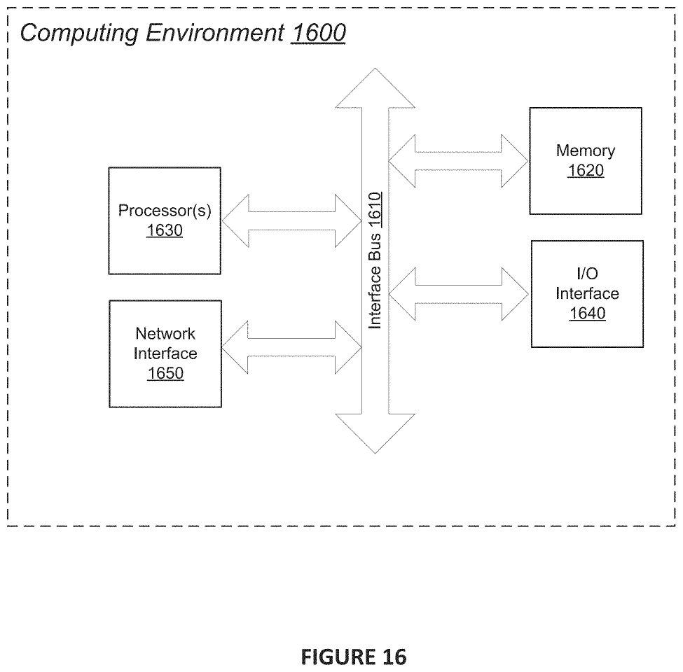

FIG. 16 depicts an example computing environment 1600 suitable for implementing aspects of the embodiments of the disclosed technology, including the control system, which can integrate one or more devices, computing, and lighting systems. As utilized herein, the phrase “computing system” generally refers to a dedicated computing device with processing power and storage memory, which supports operating software that underlies the execution of software, applications, and computer programs thereon. As used herein, an application is a small, in storage size, specialized program that is downloaded to the computing system or device. As shown by FIG. 16, computing environment 1600 includes bus 1610 that directly or indirectly couples the following components: memory 1620, one or more processors 1630, I/O interface 1640, and network interface 1650. Bus 1610 is configured to communicate, transmit, and transfer data, controls, and commands between the various components of computing environment 1600.

Computing environment 1600 typically includes a variety of computer-readable media. Computer-readable media can be any available media that is accessible by computing environment 1600 and includes both volatile and nonvolatile media, removable and non-removable media. Computer-readable media can comprise both computer storage media and communication media. Computer storage media does not comprise, and in fact explicitly excludes, signals per se.

Computer storage media includes volatile and nonvolatile, removable, and non-removable, tangible, and non-transient media, implemented in any method or technology for storage of information such as computer-readable instructions, data structures, program modules or other data. Computer storage media includes RAM; ROM; EE-PROM; flash memory or other memory technology; CD-ROMs; DVDs or other optical disk storage; magnetic cassettes, magnetic tape, magnetic disk storage or other magnetic storage devices; or other mediums or computer storage devices which can be used to store the desired information and which can be accessed by computing environment 1600.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, communication media includes wired media, such as a wired network or direct-wired connection, and wireless media, such as acoustic, RF, infrared and other wireless media. Combinations of any of the above should also be included within the scope of computer-readable media.

Memory 1620 includes computer-storage media in the form of volatile and/or nonvolatile memory. The memory can be removable, non-removable, or a combination thereof. Memory 1620 can be implemented using hardware devices such as solid-state memory, hard drives, optical-disc drives, and the like. Computing environment 1600 also includes one or more processors 1630 that read data from various entities such as memory 1620, I/O interface 1640, and network interface 1650.

I/O interface 1640 enables computing environment 1600 to communicate with different input devices and output devices. Examples of input devices include a keyboard, a pointing device, a touchpad, a touchscreen, a scanner, a microphone, a joystick, and the like. Examples of output devices include a display device, an audio device (e.g., speakers), a printer, and the like. These and other I/O devices are often connected to processor 1610 through a serial port interface that is coupled to the system bus, but can be connected by other interfaces, such as a parallel port, game port, or universal serial bus (USB). A display device can also be connected to the system bus via an interface, such as a video adapter which can be part of, or connected to, a graphics processor unit. I/O interface 1640 is configured to coordinate I/O traffic between memory 1620, the one or more processors 1630, network interface 1650, and any combination of input devices and/or output devices.

Network interface 1650 enables computing environment 1600 to exchange data with other computing devices via any suitable network. In a networked environment, program modules depicted relative to computing environment 1600, or portions thereof, can be stored in a remote memory storage device accessible via network interface 1650. It will be appreciated that the network connections shown are exemplary and other means of establishing a communications link between the computers can be used.

By way of example and without limitation, cloud computing systems can be used to perform aspects of the disclosed subject matter. Cloud-based computing generally refers to networked computer architectures where application execution, service provision, and data storage can be divided, to some extent, between clients and cloud computing devices. The “cloud” can refer to a service or a group of services accessible over a network, e.g., the Internet, by clients, server devices, and by other cloud computing systems, for example.

In one example, multiple computing devices connected to the cloud can access and use a common pool of computing power, services, applications, storage, and files. Thus, cloud computing enables a shared pool of configurable computing resources, e.g., networks, servers, storage, applications, and services, that can be provisioned and released with minimal management effort or interaction by the cloud service provider.

As an example, a cloud-based application can store copies of data and/or executable program code in the cloud computing system, while allowing client devices to download at least some of this data and program code as needed for execution at the client devices. In some examples, downloaded data and program code can be tailored to the capabilities of specific client devices, e.g., a personal computer, tablet computer, mobile phone, and/or smartphone, accessing the cloud-based application. Additionally, dividing application execution and storage between client devices and the cloud computing system allows more processing to be performed by the cloud computing system, thereby taking advantage of the cloud computing system's processing power and capability, for example.

Cloud-based computing can also refer to distributed computing architectures where data and program code for cloud-based applications are shared between one or more client devices and/or cloud computing devices on a near real-time basis. Portions of this data and program code can be dynamically delivered, as needed or otherwise, to various clients accessing the cloud-based application. Details of the cloud-based computing architecture can be largely transparent to users of client devices. By way of example and without limitation, a PC user device accessing a cloud-based application cannot be aware that the PC downloads program logic and/or data from the cloud computing system, or that the PC offloads processing or storage functions to the cloud computing system, for example.

Cloud platforms can include client-interface frontends for cloud computing systems. Such architectures can represent queues for handling requests from one or more client devices. Cloud platforms can be coupled to cloud services to perform functions for interacting with client devices. Cloud infrastructures can include service, recording, analysis, and other operational and infrastructure components of cloud computing systems. Cloud knowledge bases can be configured to store data for use by a network, and thus, cloud knowledge bases can be accessed by any of cloud services, platforms, and/or infrastructure components.

Many different types of client devices, such as devices of users, can be configured to communicate with components of network for the purpose of accessing data and executing applications provided by one or more processors and computing systems. As discussed herein any type of computing device, e.g., PC, laptop computer, tablet computer, etc., and mobile device, e.g., laptop, smartphone, mobile telephone, cellular telephone, tablet computer, etc., can be configured to transmit and/or receive data to and/or from a network.

Communication links between client devices and a network can include wired connections, such as a serial or parallel bus, Ethernet, optical connections, or other type of wired connection. Communication links can also be wireless links, such as Bluetooth, IEEE 802.11 (IEEE 802.11 can refer to IEEE 802.11-2007, IEEE 802.11n-2009, or any other IEEE 802.11 revision), CDMA, 3G, GSM, WiMAX, or other wireless based data communication links.

In other examples, the client devices can be configured to communicate with network 100 via wireless access points. Access points can take various forms. For example, an access point can take the form of a wireless access point (WAP) or wireless router. As another example, if a client device connects using a cellular air-interface protocol, such as CDMA, GSM, 3G, or 4G, an access point can be a base station in a cellular network that provides Internet connectivity via the cellular network.

As such, the client devices can include a wired or wireless network interface through which the client devices can connect to network 100 directly or via access points. As an example, the client devices can be configured to use one or more protocols such as 802.11, 802.16 (WiMAX), LTE, GSM, GPRS, CDMA, EV-DO, and/or HSPDA, among others. Furthermore, the client devices can be configured to use multiple wired and/or wireless protocols, such as “3G” or “4G” data connectivity using a cellular communication protocol, e.g., CDMA, GSM, or WiMAX, as well as for “Wi-Fi” connectivity using 802.11. Other types of communications interfaces and protocols could be used as well.

The above-described aspects of the disclosure have been described with regard to certain examples and embodiments, which are intended to illustrate but not to limit the disclosure. It should be appreciated that the subject matter presented herein can be implemented as a computer process, a computer-controlled apparatus or a computing system or an article of manufacture, such as a computer-readable storage medium.

Those skilled in the art will also appreciate that the subject matter described herein can be practiced on or in conjunction with other computer system configurations beyond those described herein, including multiprocessor systems, microprocessor-based or programmable consumer electronics, minicomputers, mainframe computers, handheld computers, personal digital assistants, e-readers, cellular telephone devices, biometric devices, mobile computing devices, special-purposed hardware devices, network appliances, and the like. The embodiments described herein can also be practiced in distributed computing environments, where tasks are performed by remote processing devices that are linked through a communications network. In a distributed computing environment, program modules can be located in both local and remote memory storage devices.

A number of different types of computing devices can be used singly or in combination to implement the resources and services in different embodiments, including general-purpose or special-purpose computer servers, storage devices, network devices, and the like. In at least some embodiments, a server or computing device that implements at least a portion of one or more of the technologies described herein, including the techniques to implement the functionality of aspects discussed herein.

Aspects

The following Aspects are illustrative only and do not limit the scope of the present disclosure or the appended claims.

Aspect 1. A computer-implemented method for detecting one or more analytes in an immunoassay, comprising: training a machine learning network using immunoassay reference data comprising sets of reference input parameters and corresponding reference analyte detection data; receiving a set of experimental input parameters, wherein the experimental input parameters comprise an identifier of an analyte; applying the machine learning network to the immunoassay reference data to identify target parameters based on the analyte; determining an immunoassay parameter set based on the target parameters, wherein the immunoassay parameter set comprises a loading concentration for at least one of the analyte and a detection reagent.

Aspect 2. The method of Aspect 1, wherein applying the machine learning network further comprises: extracting relevant immunoassay reference data based on the experimental input parameters; determining a relationship between the experimental input parameters and corresponding reference analyte detection data; and predicting target parameters for detecting the analyte.

Aspect 3. The method of Aspect 2, further comprising: classifying the relevant immunoassay reference data into variables; applying a statistical model to determine relationships between two or more variables; and training the machine learning network using the relationships between the two or more variables.

Aspect 4. The method of Aspect 3, wherein the statistical model comprises at least one of: a cost function, a logistic regression model, a multivariate regression model, and a stochastic gradient model.

Aspect 5. The method of Aspect 3, wherein the variables comprise one or more of: an antibody clonality, a protein, an antigen, analyte size, a protein source, a cell line, a tissue type, a detection sensitivity, loading concentration, a protein abundance, an antibody effect, a membrane effect, a blocking effect, an extraction effect, a protein lability, a membrane type, an analyte abundance, an antibody binding variation, an antibody clonality, a binding affinity, an antibody isoform specificity, a backbone type, a detection label, an enzyme, a detection multiplicity or a detection sensitivity, type of precast gel (e.g., a polyacrylamide gel or other gel capable of separating proteins via electrophoresis), type of membrane transferred onto, transfer method, transfer buffer, wash protocols or wash buffer.

Aspect 6. The method of Aspect 2, further comprising determining a sub-class of the analyte; identifying immunoassay reference data related to the sub-class of the analyte; determining one or more sub-class of analyte(s) of interest); and updating the target parameters based on the detected sub-class of analytes, wherein the sub-class of analytes is a transmembrane protein, a labile protein, a phosphorylation modification, a glycosylation modification, a post-translational modification, a protein form, a protein isoform, a cleaved variant of protein, or a mutant variant of protein.

Aspect 7. The method of Aspect 2, wherein the target parameters include at least one of a cell line, a lysate, and a gel type.

Aspect 8. The method of any one of Aspects 1-7, wherein immunoassay reference data comprises at least one of Western blot data, a multiplex western blot capture, quantitative data, the quantitative data optionally representative of a relative abundance of a protein, categorical data, protein detection data, and immunocytometry data.

Aspect 9. The method of Aspect 8, wherein the quantitative data comprises an analyte abundance estimate.

Aspect 10. The method of Aspect 8, wherein the immunocytometry data comprises at least one of analyte localization data or analyte intensity data.

Aspect 11. The method of any one of Aspects 1-10, wherein the analyte is at least one of a hapten, a hormone, a nucleic acid, a peptide, a modified peptide, or modified form of any of the foregoing analytes.

Aspect 12. The method of Aspect 11, wherein the modified peptide is formed from at least one of a methylation and acetylation.

Aspect 13. The method of any one of Aspects 1-12, wherein the immunoassay parameter set comprises one or more of a cell line, an analyte loading range, a target protein, detection data, a lysate type, a lysate loading concentration, a protein clonality, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type a recommended detection reagent, an antibody type, an antibody loading concentration, a protein lysate concentration, an target cell line, an target protein source, an analyte mass, a transfer condition, a validation flag, and a predicted analyte location.

Aspect 14. The method of Aspect 13, wherein the immunoassay parameter set includes a clonality recommendation, and the clonality recommendation comprises at least one of a monoclonal analyte recommendation or a polyclonal analyte recommendation.

Aspect 15. The method of Aspect 13, wherein the detection technique applies at least one of: chemiluminescence, fluorescence, enzymes, and colorimetric analysis.

Aspect 16. The method of any one of Aspects 1-15, wherein the machine learning network includes at least one of a regression model, a decision tree-based training model, and a stochastic gradient descent model.

Aspect 17. The method of any one of Aspects 1-16, wherein experimental input parameters comprise one or more of: a type of analyte, a type of protein, a clonality, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Aspect 18. The method of Aspect 17, wherein the detection data comprises chemical detection data (e.g., chem substrates), a biochemical detection data (e.g enzymes), a sequence based detection, an amplification based detection, and/or fluorescence detection data.

Aspect 19. The method of any one of Aspects 1-18, wherein the experimental input parameters comprise an analyte source, a set of proteins and a cell line.

Aspect 20. The method of any one of Aspects 1-19, wherein the experimental input parameters comprise a set of constraints received via a user interface, and the immunoassay parameter set comprises recommended values for the set of constraints.

Aspect 21. The method of any one of Aspects 1-20, wherein the experimental input parameters comprise data from a plurality of assays.

Aspect 22. The method of any one of Aspects 1-21, wherein the experimental input parameters comprise at least one detection protein or target analyte, and the immunoassay parameter set comprises a loading recommendation for at least one detection protein or target analyte.

Aspect 23. The method of any one of Aspects 1-22, wherein the experimental input parameters comprise a set of proteins and a set of constraints, received via a user interface, and the target parameters identify target values for at least one constraint in the set of constraints.

Aspect 24. The method of Aspect 23, wherein the set of constraints comprise at least one of: an available lysate, a cell line, a tissue type, a detection technology, and an antibody clonality.

Aspect 25. The method of any one of Aspects 1-24, further comprising extracting immunoassay training and reference data from images indicative of experimental immunoassay data.

Aspect 26. The method of any one of Aspects 1-25, further comprising: receiving information indicative of an immunoassay parameter set type, the immunoassay parameter set type being at least one of a troubleshooting solution or an experimental design; and updating the target parameters based on the immunoassay parameter set type.

Aspect 27. The method of Aspect 26, wherein the target parameter relates to at least one of: a Western Blot application, an immunoblotting method, a transfer method, and a type of protein gel.

Aspect 28. The method of Aspect 26, wherein the troubleshooting solution identifies one or more of an analyte source, an antibody, and dilution detection information.

Aspect 29. A system for detecting one or more analytes in an immunoassay, comprising: a at least one computing device comprising a processor and at least one memory storing instructions that when executed by the processor, causes the computing device to: receive immunoassay reference data comprising sets of input parameters and corresponding analyte detection data; receive a set of experimental input parameters, wherein the experimental input parameters comprise an analyte and a clonality; apply a machine learning network to the immunoassay reference data to identify target parameters based on the analyte; determine an immunoassay parameter set based on the target parameters; and provide the immunoassay parameter set, wherein the immunoassay parameter set comprises a loading concentration for at least one of the analyte and a detection reagent.

Aspect 30. The system of Aspect 29, wherein the at least one memory stores instructions that when executed by the processor, further causes the computing device to: extract relevant immunoassay reference data based on the experimental input parameters; determine a relationship between the experimental input parameters and corresponding reference analyte detection data; and predict target parameters for detecting the analyte.

Aspect 31. The system of Aspect 30, wherein the at least one memory stores instructions that when executed by the processor, further causes the computing device to: classify the relevant immunoassay reference data into variables; and apply a statistical model to determine relationships between two or more variables; and train the machine learning network using the relationships between the two or more variables.

Aspect 32. The system of any one of Aspects 29-31, wherein the immunoassay reference data comprises analyte detection data from a plurality of experiments comprising one or more of various loading concentrations, various detection reagents, and various antibody affinities.

Aspect 33. The system of any one of Aspects 29-32, wherein the immunoassay parameter set comprises a target range for the loading concentration for at least one of the analyte and the detection reagent.

Aspect 34. The system of any one of Aspects 29-33, wherein the immunoassay parameter set further comprises at least one of: an immunoassay performance prediction, a recommended analyte, a recommended detection reagent, an antibody concentration, a protein lysate concentration, an antibody type, an target cell line, an target protein source, an analyte mass, a transfer condition, a validation flag, and a predicted analyte location.

Aspect 35. The system of any one of Aspects 29-34, wherein the machine learning network applies at least one of a regression model, a decision tree, and a stochastic gradient descent model.

Aspect 36. The system of any one of Aspects 29-35, wherein experimental input parameters comprise one or more of: a protein, a clonality, a cell line, a detection agent, an antibody concentration, a substrate, a substrate sensitivity, a detection sensitivity, a lysate concentration, a cell line, a set of proteins, and an antibody binding variation.

Aspect 37. The system of any one of Aspects 29-36, wherein the instructions further cause the computing device to extract immunoassay reference data from images representative of experimental immunoassay data.

Aspect 38. The system of any one of Aspects 29-37 wherein the system further comprises a user interface, and the user interface comprises at least one of: an instrument console, a web tool, a graphical user interface, and a display on a computing device.

Aspect 39. A non-transitory computer-readable medium for storing instructions that, when executed by one or more processors, cause a device to perform the methods of any one of Aspects 1-30.

Aspect 40. A device comprising: one or more processors; and a memory storing instructions that, when executed by the one or more processors, cause the device to perform the methods of any one of Aspects 1-39.

Aspect 41. A computer-implemented method for operating an immunoassay instrument support apparatus, comprising: receiving immunoassay reference data comprising sets of reference input parameters and corresponding reference analyte detection data; receiving a set of experimental input parameters, the experimental input parameters comprising at least one variable related the immunoassay experiment; receiving information indicative of an issue with at least one of: the immunoassay reference data, an experimental input parameter, and a result of immunoassay experiment; applying a machine learning network to the immunoassay reference data to identify target parameters based on the issue; and determining an immunoassay parameter set to resolve based on the target parameters.

Aspect 42. The method of Aspect 41, wherein the issue is at least one of an analyte detection issue or an immunoassay reference data extraction issue.

Aspect 43. The method of Aspect 42, wherein the issue relates to extraction of immunoassay reference data from images indicative of experimental immunoassay data.

Aspect 44. The method of any one of Aspects 41-43, wherein the immunoassay parameter set provides at least one of: a protein lysate from a cell line source, a type of lysate, an antibody dilution range for target detection, an antibody dilution based on a clonality and host backbone type, a type of detection reagent, a type of detection technology, and an target detection technology to ensure linearity.

Aspect 45. The method of any one of Aspects 41-44, wherein the experimental input parameters comprise at least one of: a type of analyte, a type of protein, a clonality, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, and protein mass ranges.

Aspect 46. The method of any one of Aspects 41-45, wherein the experimental input parameters comprise one or more of: a hapten, a hormone, a modified nucleic acid, a peptide, an antibody clonality, a protein, an antigen, analyte size, a protein source, a cell line, a tissue type, a detection sensitivity, loading concentration, a protein abundance, an antibody effect, a membrane effect, a blocking effect, an extraction effect, a protein lability, a membrane type, an analyte abundance, an antibody binding variation, an antibody clonality, a binding affinity, an antibody isoform specificity, a backbone type, a detection label, an enzyme, a detection multiplicity or a detection sensitivity.

Aspect 47. The method of any one of Aspects 41-46, wherein the experimental input parameters comprise a set of variables received via a user interface, the issue relates to detection, and the immunoassay parameter set comprises recommended values for the set of variables.

Aspect 48. The method of any one of Aspects 41-47, wherein immunoassay parameter set comprises one or more of: a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity or a detection sensitivity.

Aspect 49. The method of any one of Aspects 41-48, wherein the immunoassay parameter set comprises one or more of: an target range for the loading concentration for at least one of the analyte, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a recommended detection reagent, an antibody type, an antibody loading concentration, a protein lysate concentration, an target protein source, an analyte mass, a transfer condition, a validation flag, and a predicted analyte location.

Aspect 50. The method of any one of Aspects 41-49, wherein the machine learning network applies at least one of a regression model, a decision tree-based training model, and a stochastic gradient descent model.

Aspect 51. A computer-implemented method for operating an immunoassay instrument support apparatus, comprising: receiving immunoassay reference data comprising sets of reference input parameters and corresponding reference analyte detection data; receiving a set of experimental input parameters; receiving at least one target variable for to the immunoassay experiment; applying a machine learning network to the immunoassay reference data to identify target parameters based on the target variable; and determining an immunoassay parameter set for obtaining the target variable based on the target parameters, wherein the target variable is an analyte.

Aspect 52. The method of Aspect 51, wherein the set of experimental input parameters represent a method of experimentation.

Aspect 53. The method of any one of Aspects 51-52, wherein the method of experimentation is a Western Blot method, an immunoblotting method, a transfer method, and method utilizing protein gel.

Aspect 54. The method of any one of Aspects 51-53, wherein the at least one target variable comprises one or more of: a type of analyte, a type of protein, a clonality, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, and protein mass ranges.

Aspect 55. The method of any one of Aspects 51-54, wherein the at least one target variable comprises one or more of: a hapten, a hormone, a modified nucleic acid, a peptide, a protein, an antigen, analyte size, a protein source, a cell line, a tissue type, a detection sensitivity, loading concentration, a protein abundance, an antibody effect, a membrane effect, a blocking effect, an extraction effect, a protein lability, a membrane type, an analyte abundance, an antibody binding variation, an antibody clonality, a binding affinity, an antibody isoform specificity, a backbone type, a detection label, an enzyme, a detection multiplicity or a detection sensitivity.

Aspect 56. The method of any one of Aspects 51-55, wherein the immunoassay parameter set comprises one or more of: a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity or a detection sensitivity.

Aspect 57. The method of any one of Aspects 51-56, wherein the immunoassay parameter set comprises one or more of: an target range for the loading concentration for at least one of the analyte, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a recommended detection reagent, an antibody type, an antibody loading concentration, a protein lysate concentration, an target protein source, an analyte mass, a transfer condition, a validation flag, and a predicted analyte location.

Aspect 58. The method of any one of Aspects 51-57, wherein the machine learning network applies at least one of a regression model, a decision tree-based training model, and a stochastic gradient descent model.

Aspect 59. A computer-implemented method for operating an immunoblot instrument support apparatus, comprising: receiving immunoblot reference data comprising sets of reference input parameters and corresponding reference analyte detection data; receiving a set of experimental input parameters, wherein the experimental input parameters comprise an analyte; applying a machine learning network to the immunoblot reference data to identify target parameters based on the analyte; and determining an immunoassay parameter set based on the target parameters, wherein the immunoassay parameter set comprises a loading concentration for at least one of the analyte and a detection reagent.

Aspect 60. The method of Aspect 59, wherein the immunoassay parameter set comprises one or more of a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type a recommended detection reagent, an antibody type, an antibody loading concentration, a protein lysate concentration, an target protein source, an analyte mass, a transfer condition, a validation flag, and a predicted analyte location.

Aspect 61. The method of any one of Aspects 59-60, wherein experimental input parameters comprise one or more of: a type of analyte, a type of protein, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Aspect 62. A computer-implemented method for operating an immunoassay instrument support apparatus, comprising: receiving immunoassay reference data comprising sets of reference input parameters and corresponding reference analyte detection data; receiving a set of experimental input parameters, wherein the experimental input parameters comprise an analyte; applying a machine learning network to the immunoassay reference data to identify target parameters, including a set of multiple proteins for profiling, based on the analyte; and determining an immunoassay parameter set for profiling the set of multiple proteins based on the target parameters, wherein the immunoassay parameter set comprises a loading concentration for at least one of the analyte and a detection reagent.

Aspect 63. The computer implemented method of Aspect 62, wherein the immunoassay is a bead-based immunoassay.

Aspect 64. The computer implemented method of Aspect 63, wherein the bead-based immunoassay is at least one of a multiplex assay, a bead-based immunoassay utilizing panels, or a bead-based immunoassay utilizing activated surfaces panel builder.

Aspect 65. The method of any one of Aspects 62-64, wherein the immunoassay parameter set comprises one or more of a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte, a clonality, an antibody type, the detection reagent, an immunoassay performance prediction, a recommended protein source, a cell source, an antibody clonality, a detection technique, a clonality recommendation, an antibody recommendation, an analyte recommendation, an analyte source recommendation, a gel type a recommended detection reagent, an antibody type, an antibody loading concentration, a protein lysate concentration, an target cell line, an target protein source, an analyte mass, a transfer condition, a validation flag, and a predicted analyte location.

Aspect 66. The method of any one of Aspects 62-65, wherein experimental input parameters comprise one or more of: a type of analyte, a type of protein, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Aspect 67. A computer-implemented method for operating an immunoassay instrument support apparatus, comprising: receiving immunoassay reference data comprising sets of reference input parameters and corresponding reference analyte detection data; receiving a set of experimental input parameters, wherein the experimental input parameters comprise an analyte, and single cell information (e.g., information related to an individual or sole cell); applying a machine learning network to the immunoassay reference data to identify target parameters based on the analyte; and determining an immunoassay parameter set for detecting multiple proteins in the single cell based on the target parameters, wherein the immunoassay parameter set comprises a loading concentration for at least one of the analyte and a detection reagent.

Aspect 68. The method of Aspect 67, wherein the immunoassay parameter set comprises one or more of a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type a recommended detection reagent, an antibody type, an antibody loading concentration, a protein lysate concentration, an target protein source, an analyte mass, a transfer condition, a validation flag, and a predicted analyte location.

Aspect 69. The method of any one of Aspects 67-68, wherein experimental input parameters comprise one or more of: a type of analyte, a type of protein, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Aspect 70. A computer-implemented method for operating an immunoassay instrument support apparatus for flow-based immunoassays, comprising: receiving immunoassay reference data comprising sets of reference input parameters and corresponding reference analyte detection data, wherein the immunoassay reference data comprises at least one set of flow-based immunoassay data; receiving a set of experimental input parameters, wherein the experimental input parameters comprise an analyte, and single cell information; applying a machine learning network to the immunoassay reference data, including the flow-based immunoassay data to identify target parameters based on the analyte; and determining an immunoassay parameter set for detecting multiple proteins in the single cell based on the target parameters, wherein the immunoassay parameter set comprises a loading concentration for at least one of the analyte and a detection reagent. Example flow assays include, e.g., a flow assay that uses colored particles, a competitive assay, and the like.

Aspect 71. The method of Aspect 70, wherein the flow-based immunoassay is a lateral flow immunoassay.

Aspect 72. The method of any one of Aspects 70-71, wherein the immunoassay parameter set comprises one or more of a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type a recommended detection reagent, an antibody type, an antibody loading concentration, a protein lysate concentration, an target protein source, an analyte mass, a transfer condition, a validation flag, and a predicted analyte location.

Aspect 73. The method of any one of Aspects 70-72, wherein experimental input parameters comprise one or more of: a type of analyte, a type of protein, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Aspect 74. A computer-implemented method for optimizing fluorescent analyte detection in immunoassays, comprising: receiving reference data comprising sets of reference input parameters and corresponding reference analyte detection data; receiving a set of experimental input parameters, wherein the experimental input parameters comprise an analyte; applying a machine learning network to the reference data to identify target parameters for fluorescent detection based on the analyte; and determining an immunoassay parameter set based on the target parameters, wherein the immunoassay parameter set comprises a loading concentration for at least one of the analyte and a detection reagent.

Aspect 75. The method of Aspect 74, wherein the immunoassay parameter set further comprises a type of dye for detecting at least one of proteins or analytes.

Aspect 76. The method of Aspect 75, wherein the type of dye is at least one of an Ab-conjugate, a secondary Ab conjugate, or a strong dye to detect a low abundance analyte.

Aspect 77. The method of any one of Aspects 74-76, wherein the immunoassay parameter set comprises one or more of a cell line, an analyte loading range, a target protein, detection data, a lysate type, a lysate loading concentration, an antibody type, an antibody binding site, an antibody clonality, an antibody dilution range, a binding affinity, an antibody isoform specificity, a backbone type, a protein stability, a protein lability, a detection label, an enzyme, a detection reagent, a detection multiplicity, a detection sensitivity, an target range for the loading concentration for at least one of the analyte, a clonality, an antibody type, the detection reagent, an immunoassay performance prediction, a recommended protein source, an optimum cell line, a cell source, an antibody clonality, a detection technique, a clonality recommendation, an antibody recommendation, an analyte recommendation, an analyte source recommendation, a gel type a recommended detection reagent, an antibody type, an antibody loading concentration, a protein lysate concentration, an target protein source, an analyte mass, a transfer condition, a validation flag, and a predicted analyte location.

Aspect 78. The method of any one of Aspects 74-77, wherein experimental input parameters comprise one or more of: a type of analyte, a type of protein, a cell line, a detection reagent, an antibody concentration, a substrate, a substrate sensitivity, a detection sensitivity, a lysate concentration, a cell line, a set of proteins, an antibody binding variation, blocking data, cell lysate preparation data, protein lability, protein stability, gel parameters, analyte mass ranges, protein mass ranges, a cell line, detection data, a lysate type, a lysate loading concentration, a protein, a protein isoform, a fragment of a protein or a post-translationally modified protein, an antibody binding site, an antibody clonality, an antibody dilution, a binding affinity, an antibody isoform specificity, a backbone type, a protein stability, a protein lability, a detection label, an enzyme, a detection multiplicity or a detection sensitivity.

Aspect 79. A method, comprising: receiving experimental data corresponding to an immunoassay experiment, wherein the experimental data is based on an analyte of interest; analyzing, by a machine learning process trained to determine a degree that a band of the analyte shifts in immunoassays, the experimental data; and determining, based on the analyzing, the degree of shift for a band in the immunoassay experiment, wherein the band comprises the analyte of interest.

Aspect 80. The method of Aspect 79, wherein the machine learning process comprises a neural network.

Aspect 81. The method of Aspect 80, wherein the neural network is a feedforward network or a deep neural network.

Aspect 82. The method of Aspect 79, wherein: the analyzing comprises analyzing an immunoassay image of the immunoassay experiment; and the determining comprises marking the degree of shift for the band on the immunoassay image.

Aspect 83. The method of Aspect 82, wherein the immunoassay image comprises a stained gel or a capillary gel where the analyte is loaded.

Aspect 84. The method of Aspect 79, wherein the immunoassay experiment is a bead-based immunoassay or a flow-based immunoassay.

Aspect 85. The method of Aspect 79, wherein the analyte is at least one of a protein, a hapten, a hormone, a nucleic acid, a peptide, a modified peptide, or a modified form of any of the foregoing analytes.

Aspect 86. The method of Aspect 79, wherein the machine learning process has been trained using an immunoassay dataset comprising at least one of glycosylation, disulfide bonds, modified residue, 3-(N-morpholino)propanesulfonic acid (MOPS), ubiquitination, lipidation, 2-(N-morpholino)ethanesulfonic acid (MES), isoelectric point (pI), SUMOylation, Tris acetate, interchain or polymer cross links, gel type, buffer type, or degree of shift.

Aspect 87. The method of Aspect 79 or Aspect 86, wherein the experimental data comprises at least one of glycosylation, disulfide bonds, modified residue, 3-(N-morpholino)propanesulfonic acid (MOPS), ubiquitination, lipidation, 2-(N-morpholino)ethanesulfonic acid (MES), isoelectric point (pI), SUMOylation, Tris acetate, or interchain or polymer cross links for the analyte of interest and gel type and buffer type for the immunoassay experiment.

Aspect 88. The method of Aspect 79, wherein the degree of shift of the band is caused by a shift in the molecular weight corresponding to the analyte of interest.

Aspect 89. The method of Aspect 79, further comprising: determining whether the experimental data should be modified; and displaying the determined modifications.

Aspect 90. A system, comprising: a processor; and a memory coupled to the processor, the memory having instructions stored thereon that, when executed by the processor, cause the processor to perform operations comprising: receiving experimental data, wherein the experimental data comprises an identifier of an analyte and an immunoassay image; analyzing the immunoassay image to mark features of the immunoassay image, wherein the features comprises a band; determining, by a machine learning process, a degree of shift for a band on the immunoassay image, wherein the band comprises an analyte of interest that corresponds to the identifier; and displaying the degree of shift for the band on the immunoassay image.

Aspect 91. The system of Aspect 90, wherein the machine learning process comprises a neural network.

Aspect 92. The system of Aspect 91, wherein the neural network is a feedforward network or a deep neural network.

Aspect 93. The system of Aspect 90, wherein the processor performs further operations comprising: calculating a molecular weight data for the band; and displaying a data table, wherein the data table comprises the calculated molecular weight.

Aspect 94. The system of Aspect 90, wherein the experimental data further comprises at least one identifier of a cell line, an identifier of a molecular marker, a lysate type, a loading concentration, and a gel type.

Aspect 95. The system of Aspect 90, wherein the processor performs further operations comprising: determining whether the experimental data should be modified; and displaying the determined modifications.

Aspect 96. The system of Aspect 90, wherein the immunoassay image is an image of a bead-based immunoassay or a flow-based immunoassay.

Aspect 97. The system of Aspect 90, wherein the analyte is at least one of a protein, a hapten, a hormone, a nucleic acid, a peptide, a modified peptide, or a modified form of any of the foregoing analytes.

Aspect 98. The system of Aspect 90, wherein the features further comprise at least one of a frame of the immunoassay image and a lane of the immunoassay image.

Aspect 99. The system of Aspect 90, wherein the immunoassay image comprises a plurality of bands.

Aspect 100. The system of Aspect 98, wherein the immunoassay image comprises a plurality of lanes.

Aspect 101. The system of Aspect 99, wherein each lane in the plurality of lanes is analyzed to determine the shift of the band in each lane.

Aspect 102. The system of Aspect 90, wherein the processor performs operations further comprising: displaying whether the band was found, found with non-specific bands, not found, or there were no bands.

Aspect 103. The system of Aspect 90, wherein the machine learning process has been trained with an immunoassay dataset to determine a degree that a band of an analyte shifts in an immunoassay experiment.

Aspect 104. The system of Aspect 103, wherein the immunoassay dataset comprises at least one glycosylation, disulfide bonds, modified residue, 3-(N-morpholino)propanesulfonic acid (MOPS), ubiquitination, lipidation, 2-(N-morpholino)ethanesulfonic acid (IVIES), isoelectric point (pI), SUMOylation, Tris acetate, interchain or polymer cross links, gel type, and buffer type.

Aspect 105. The system of Aspect 90, wherein the receiving and displaying are performed by a user interface.

1-78. (canceled)

79. A method, comprising:
acquiring experimental data corresponding to an immunoassay experiment, wherein the experimental data is based on an analyte of interest;
analyzing, by a machine learning process trained to determine a degree that a band of the analyte shifts in immunoassays, the experimental data; and
determining, based on the analyzing, the degree of shift for a band in the immunoassay experiment, wherein the band comprises the analyte of interest.

80. The method of claim 79, wherein the machine learning process comprises a neural network, wherein the neural network comprises a feedforward network or a deep neural network.

81. (canceled)

82. The method of claim 79, wherein:
the analyzing comprises analyzing an immunoassay image of the immunoassay experiment; and
the determining comprises marking the degree of shift for the band on the immunoassay image,
wherein the immunoassay image comprises a stained gel or a capillary gel where the analyte is loaded.

83. (canceled)

84. The method of claim 79, wherein the immunoassay experiment is a bead-based immunoassay or a flow-based immunoassay.

85. The method of claim 79, wherein the analyte is at least one of a protein, a hapten, a hormone, a nucleic acid, a peptide, a modified peptide, or a modified form of any of the foregoing analytes.

86. The method of claim 79, wherein the machine learning process has been trained using an immunoassay dataset comprising at least one of glycosylation, disulfide bonds, modified residue, 3-(N-morpholino)propanesulfonic acid (MOPS), ubiquitination, lipidation, 2-(N-morpholino)ethanesulfonic acid (MES), isoelectric point (pI), SUMOylation, Tris acetate, interchain or polymer cross links, gel type, buffer type, or degree of shift.

87. The method of claim 79 or claim 86, wherein the experimental data comprises at least one of glycosylation, disulfide bonds, modified residue, 3-(N-morpholino)propanesulfonic acid (MOPS), ubiquitination, lipidation, 2-(N-morpholino)ethanesulfonic acid (MES), isoelectric point (pI), SUMOylation, Tris acetate, or interchain or polymer cross links for the analyte of interest and gel type and buffer type for the immunoassay experiment.

88. The method of claim 79, wherein the degree of shift of the band is caused by a shift in the molecular weight corresponding to the analyte of interest.

89. The method of claim 79, further comprising:
determining whether the experimental data should be modified; and
displaying the determined modifications.

90. A system, comprising:
at least one processor; and
a memory coupled to the at least one processor, the memory having instructions stored thereon that, when executed by the processor, cause the processor to perform operations comprising:
acquiring experimental data, wherein the experimental data comprises an identifier of an analyte and an immunoassay image;
analyzing the immunoassay image to mark features of the immunoassay image, wherein the features comprises at least one band;
determining, by a machine learning process, a degree of shift for a band on the immunoassay image, wherein the band comprises an analyte of interest that corresponds to the identifier; and
displaying the degree of shift for the band on the immunoassay image.

91. The system of claim 90, wherein the machine learning process comprises a neural network.

92. (canceled)

93. The system of claim 90, wherein the processor performs further operations comprising:
calculating a molecular weight data for the band; and
displaying a data table, wherein the data table comprises the calculates molecular weight.

94. The system of claim 90, wherein the experimental data further comprises at least one identifier of a cell line, an identifier of a molecular marker, a lysate type, a loading concentration, and a gel type.

95. The system of claim 90, wherein the processor performs further operations comprising:
determining whether the experimental data should be modified; and
displaying the determined modifications.

96. The system of claim 90, wherein the immunoassay image is an image of a bead-based immunoassay or a flow-based immunoassay.

97. The system of claim 90, wherein the analyte is at least one of a protein, a hapten, a hormone, a nucleic acid, a peptide, a modified peptide, or a modified form of any of the foregoing analytes.

98. The system of claim 90, wherein the features further comprise at least one of a frame of the immunoassay image and at least one lane of the immunoassay image, wherein the at least one lane is analyzed to determine the shift of the band in each lane.

99-101. (canceled)

102. The system of claim 90, wherein the at least one processor performs operations further comprising:
displaying whether the band was found, found with non-specific bands, not found, or there were no bands.

103. The system of claim 90, wherein the machine learning process has been trained with an immunoassay dataset to determine a degree that a band of an analyte shifts in an immunoassay experiment.

104. The system of claim 103, wherein the immunoassay dataset comprises at least one glycosylation, disulfide bonds, modified residue, 3-(N-morpholino)propanesulfonic acid (MOPS), ubiquitination, lipidation, 2-(N-morpholino)ethanesulfonic acid (MES), isoelectric point (pI), SUMOylation, Tris acetate, interchain or polymer cross links, gel type, and buffer type.

105. (canceled)
